SEQUENCE LISTING (2 sequences)

SEQ ID NO: 1 - protein, 4 aa, synthetic construct
TKPR

SEQ ID NO: 2 - protein, 6 aa, synthetic construct
TKPRGY

(19) United States
(12) Patent Application Publication
(10) Pub. No.: US 2025/0025561 A1
(43) Pub. Date: Jan. 23, 2025

(54) LIQUID COMPOSITIONS COMPRISING A PHOSPHORYLCHOLINE-TUFTSIN CONJUGATE

(71) Applicant: Tarsier Pharma Ltd., Zichron Yaacov (IL)

(72) Inventors: Chaim EIDELMAN, Tal El (IL); Zohar MILMAN-LEVINSON, Kibbutz Tel-Yosef (IL); Daphne HAIM-LANGFORD, Zichron Yaacov (IL); Sharon NAVON, Kiryat Ata (IL); Galit LEVIN, Even-Yehuda (IL)

(73) Assignee: Tarsier Pharma Ltd., Zichron Yaacov (IL)

(21) Appl. No.: 18/711,977

(22) PCT Filed: Nov. 21, 2022
(86) PCT No.: PCT/IL2022/051240

§ 371 Date: May 21, 2024

Related U.S. Application Data
(60) Provisional application No. 63/281,690, filed on Nov. 21, 2021.

(51) Int. Cl. A61K 47/54; A61K 38/07; A61P 31/10

(52) U.S. Cl. CPC A61K 47/548; A61K 38/07; A61P 31/10

(57) ABSTRACT

Provided herein is a fungicidal composition, comprising a pharmaceutically effective amount of a phosphorylcholine-tuftsin conjugate, and optionally comprising a pharmaceutically acceptable carrier. A method of using the fungicidal composition such as for treating or preventing a disease or disorder associated with fungal infection in a subject in need thereof is also provided. Furthermore, method of using the fungicidal composition such as for enhancing shelf-life and/or preventing fungal contamination of food-products is also provided.

### CROSS REFERENCE TO RELATED APPLICATIONS

This application is a National Phase of PCT Patent Application No. PCT/IL2022/051240 having International filing date of Nov. 21, 2022, which claims the benefit of priority of U.S. Provisional Patent Application No. 63/281,690 filed on Nov. 21, 2021, the contents of which are hereby incorporated by reference in their entirety.

### REFERENCE TO AN ELECTRONIC SEQUENCE LISTING

The contents of the electronic sequence listing (TARS-P-004-PCT SQL.xml; Size: 2,647 bytes; and Date of Creation: Nov. 21, 2022) is herein incorporated by reference in its entirety.

### FIELD OF INVENTION

This invention is directed to novel phosphorylcholine-tuftsin conjugates and fungicidal compositions comprising thereof. Furthermore, the invention in some embodiments thereof, is directed to use of the phosphorylcholine-tuftsin conjugates and/or of the fungicidal compositions as a preservative, or for prevention or treatment of fungal infections within a subject and/or conditions associated therewith.

### BACKGROUND OF THE INVENTION

Numerous fungicides are well known in the art. While having satisfactory antifungal activity, these compounds are quite toxic. Accordingly, there is a need for new fungicidal compounds characterized by low toxicity. Furthermore, there is a need for new non-toxic compounds which can be utilized as spoilage inhibitors, such as in liquid formulations and/or in food products.

Tuftsin-PhosphorylCholine (TRS) is bi-specific small molecule with immunomodulatory activities. Tuftsin (Thr-Lys-Pro-Arg) is a natural immunomodulating peptide produced by enzymatic cleavage of the Fc-domain of the heavy chain of IgG in the spleen. Phosphorylcholine (PC) is a small zwitterionic molecule secreted by helminths which permits helminths to survive in the host inducing a situation of immune tolerance as well as on the surface of some bacteria and apoptotic cells. Furthermore, TRS is known for its toll-like receptor (TLR-4) inhibitory activity.

### SUMMARY OF THE INVENTION

In one aspect of the invention, there is provided a fungicidal composition comprising a fungicidal effective amount of a phosphorylcholine-tuftsin conjugate, a salt thereof, a functional derivative thereof, or any combination thereof.

In one embodiment, the phosphorylcholine-tuftsin conjugate comprises at least one phosphorylcholine moiety or a derivative thereof and tuftsin or a derivative thereof covalently linked via a spacer.

In one embodiment, the spacer comprises at least two amino acids.

In one embodiment, the phosphorylcholine moiety or a derivative thereof is covalently linked to said spacer via a diazo group.

In one embodiment, the phosphorylcholine-tuftsin conjugate is represented by Formula 1:

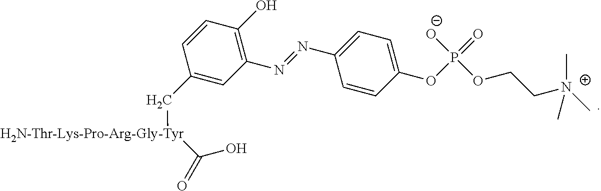

In one embodiment, the fungicidal composition of the invention further comprises a suitable carrier.

In one embodiment, the fungicidal composition is a pharmaceutical composition comprising a pharmaceutically acceptable carrier.

In one embodiment, a w/w of the carrier within the fungicidal composition is between 10 and 99.99%.

In one embodiment, the fungicidal composition is in a form of a liquid formulation.

In another aspect, there is provided a method for treating or preventing a disease or disorder associated with a fungal infection in a subject in need thereof, the method comprising administering to said subject a therapeutically effective amount of the fungicidal composition of the invention.

In one embodiment, the therapeutically effective amount is sufficient for reducing fungal load within the subject, or for ameliorating at least one symptom associated with said disease or said disorder within the subject.

In another aspect, there is provided a method for reducing or inhibiting activity or growth of at least one fungus, comprising contacting a fungus with the fungicidal effective amount of the fungicidal composition of the invention, thereby reducing activity, or reducing growth of said fungus.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the study of the following detailed description.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE FIGURES

FIG. 1 is a graph representing fermentation activity of aqueous yeast (S. cerevisiae) suspension as a function of the concentration of phosphorylcholine tuftsin conjugate of Formula 1, as compared to a control suspension without the phosphorylcholine tuftsin conjugate.

FIG. 2 is a graph representing fermentation activity of aqueous yeast (S. cerevisiae) suspension as a function of the concentration of phosphorylcholine tuftsin conjugate of Formula 2, as compared to a control suspension without the phosphorylcholine tuftsin conjugate.

### DETAILED DESCRIPTION OF THE INVENTION

The present invention, in some embodiments thereof, relates to a fungicidal composition comprising a phosphorylcholine-tuftsin conjugate of Formula 1 (hereinafter “TRS”), and to a method for treating a medical condition (e.g., a disease or disorder) within a subject associated with fungal infection, by administering a therapeutically effective amount of the fungicidal composition to the subject in need thereof. The present invention, in further embodiments thereof, relates to anti-fungal (or anti-mold) compositions for use in the prevention or reduction of fungal loading within a composition (e.g. an aqueous composition, or a food matter).

The present invention, in some embodiments thereof, is based on a surprising finding that phosphorylcholine-tuftsin conjugates disclosed herein were able to substantially inhibit yeast function (fermentation) in an aqueous suspension comprising yeast and nutrients (e.g. sucrose).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Phosphorylcholine-Tuftsin Conjugates

In one aspect of the invention, there is provided (i) a phosphorylcholine tuftsin conjugate, (ii) a functional derivative thereof, including any salt or any combination of (i) and (ii), wherein the phosphorylcholine tuftsin conjugate is represented by Formula 2:

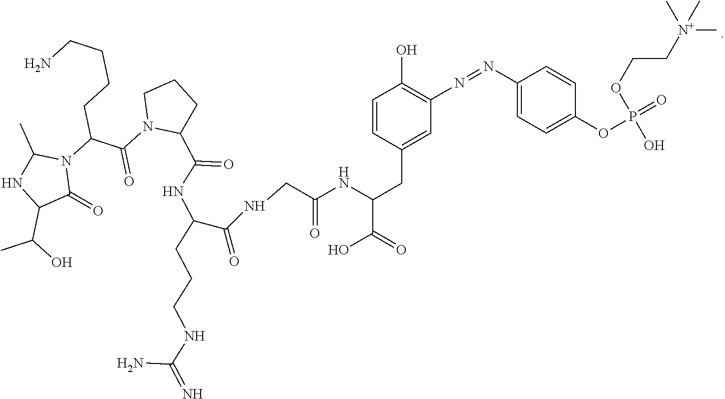 [SMILES: CC(O)C1NC(C)N(C(CCCCN)C(=O)N2CCCC2C(=O)NC(CCCNC(=N)N)C(=O)NCC(=O)NC(Cc2ccc(O)c(N=Nc3ccc(OP(=O)(O)OCC[N+](C)(C)C)cc3)c2)C(=O)O)C1=O]

In some embodiments, the functional derivative comprises any phosphorylcholine-tuftsin conjugate having at least 80%, at least 85%, at least 90%, at least 95%, at least 97%, or between 80 and 99%, between 85 and 99%, between 90 and 99%, or between 95 and 99% structure similarity to the phosphorylcholine-tuftsin conjugate of Formula 2, and characterized by an antifungal activity as disclosed hereinbelow.

In some embodiments, the term “structure similarity” refers to a fingerprint similarity between two molecules. The term “fingerprint similarity” is well-understood by a skilled artisan. In some embodiments, the fingerprint similarity is calculated based on circular fingerprints, substructure keys-based fingerprints, and/or topological or path-based fingerprints.

Exemplary circular fingerprints include but are not limited to: Molprint 2D, ECFP (or Morgan fingerprint), FCFP, etc. In some embodiments, the term “structure similarity” as used herein, is calculated by Morgan fingerprint.

In some embodiments, the functional derivative is represented by Formula 3:

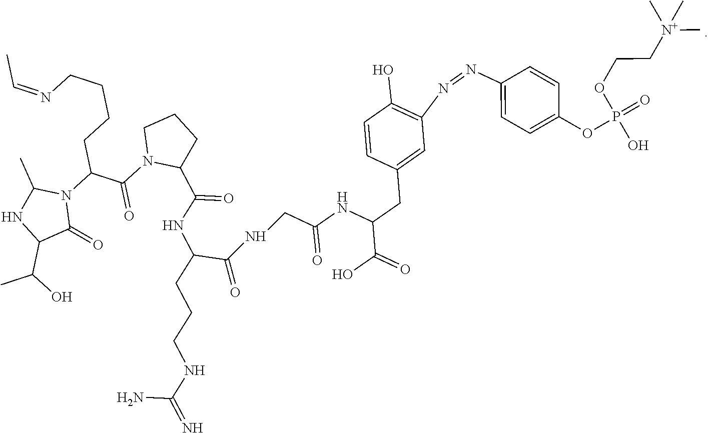 [SMILES: C/C=N\CCCCC(C(=O)N1CCCC1C(=O)NC(CCCNC(=N)N)C(=O)NCC(=O)NC(Cc1ccc(O)c(/N=N\c2ccc(OP(=O)(O)OCC[N+](C)(C)C)cc2)c1)C(=O)O)N1C(=O)C(C(C)O)NC1C]

In some embodiments, the functional derivative is represented by Formula 4:

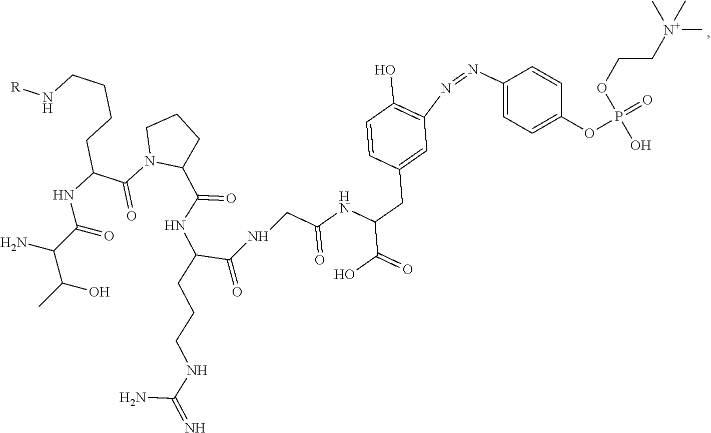

### or by Formula 5:

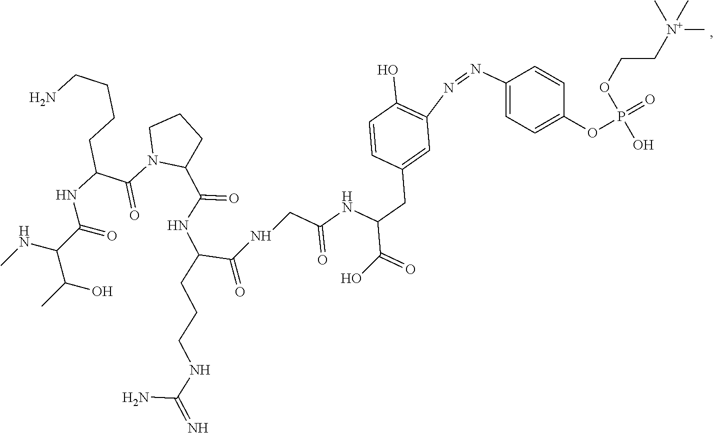

wherein R is selected from

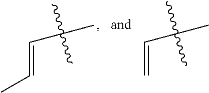

including any salt or a deprotonated amine thereof, as allowed by valency.

In some embodiments, the phosphorylcholine tuftsin conjugate, and/or the functional derivative of the phosphorylcholine tuftsin conjugate encompass any salt thereof, wherein the salt is a pharmaceutically acceptable salt.

As used herein, the term “pharmaceutically acceptable salt” refers to any non-toxic salt of a compound of the present invention that, upon administration to a subject, e.g., a human, is capable of providing, either directly or indirectly, a compound of this invention or an active metabolite or residue thereof. For example, the term “pharmaceutically acceptable” can mean approved by a regulatory agency of the Federal or a state government or listed in the U. S. Pharmacopeia or other generally recognized pharmacopeia for use in animals, and more particularly in humans.

Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Non-limiting examples of pharmaceutically acceptable salts include but are not limited to: acetate, trifluoroacetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

Additional examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N+(C1-4alkyl) 4 salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

In some embodiments, the phosphorylcholine tuftsin conjugates described herein are chiral compounds (i.e. possess an asymmetric carbon atom). In some embodiments, diastereomers, geometric isomers and individual isomers are encompassed within the scope of the present invention. In some embodiments, the phosphorylcholine tuftsin conjugate described herein is in form of a racemic mixture. In some embodiments, phosphorylcholine tuftsin conjugate is in form of a single enantiomer, or of a single diastereomer.

In some embodiments, the phosphorylcholine tuftsin conjugate is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the phosphorylcholine tuftsin conjugate is represented by Formula 2A:

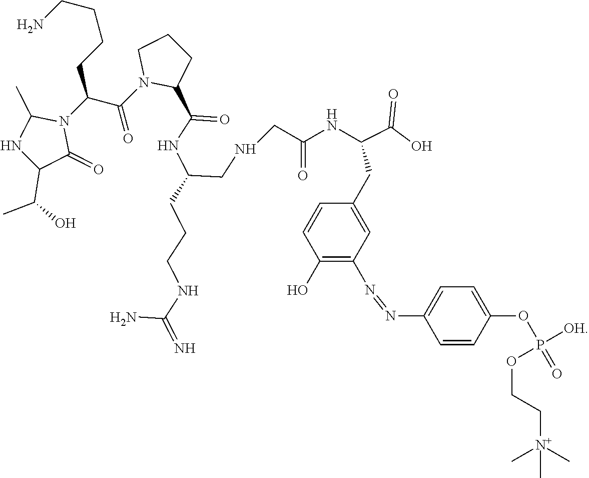

In some embodiments, the functional derivative is represented by Formula 3A:

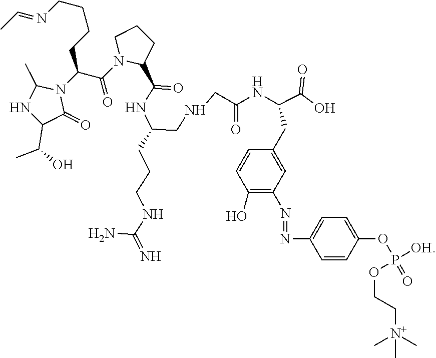

In some embodiments, the phosphorylcholine tuftsin conjugate comprising an unsaturated bond is in a form of a trans-, or cis-isomer. In some embodiments, the phosphorylcholine tuftsin conjugate comprises a mixture of cis- and trans-isomers, as described hereinabove.

In some embodiments, the phosphorylcholine tuftsin conjugate described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. Therefore, single stereochemical isomers as well as enantiomeric, diastereomeric, geometric, conformational, and rotational mixtures of the present compounds are within the scope of the invention.

Unless otherwise indicated, all tautomeric forms of the phosphorylcholine tuftsin conjugate of the invention are within the scope of the invention.

In some embodiments, the phosphorylcholine tuftsin conjugate of the invention is a single compound characterized by a chemical purity of at least 90%, of at least 92%, at least 95%, at least 96%, at least 97%, at least 99%, at least 99.5%, between 90 and 99%, between 92 and 95%, between 92 and 97%, between 92 and 99%, including any range between. Chemical purity can be determined by an analytical HPLC, GC, or by LC-MS.

In some embodiments, the phosphorylcholine tuftsin conjugate of the invention comprises less than 0.5%, less than 0.1%, less than 0.01% of phosphorylcholine tuftsin conjugate of Formula 1. In some embodiments, the phosphorylcholine tuftsin conjugate of the invention is devoid of detectable trace amounts of phosphorylcholine tuftsin conjugate of Formula 1, wherein detectable is as determined by standard methods of analysis, such as nuclear magnetic resonance (NMR), high performance liquid chromatography (HPLC) and mass spectrometry (MS), gas-, liquid-chromatography mass spectrometry (GC-MS, LC-MS), and similar, used by those of skill in the art to assess such purity.

In some embodiments, the phosphorylcholine tuftsin conjugate of the invention encompasses a plurality (e.g. 2, 3, 4, or 5) of chemically distinct compounds (e.g. plurality of conjugates of Formulae 1-5).

### Fungicidal Composition

In another aspect of the invention, there is provided a composition comprising one or more of the phosphorylcholine-tuftsin conjugates of the invention and a carrier. In some embodiments, the carrier is a liquid carrier. In some embodiments, the carrier is a pharmaceutically acceptable carrier. In some embodiments, the composition is a pharmaceutical composition, or a fungicidal composition.

In another aspect of the invention, there is provided a fungicidal composition comprising one or more of the phosphorylcholine-tuftsin conjugates of the invention. In some embodiments, the fungicidal composition comprises a fungicidal effective amount of one or more phosphorylcholine-tuftsin conjugates of the invention (i.e., the conjugates of Formulae 1-5). In some embodiments, the fungicidal composition comprises a fungicidal effective amount of the phosphorylcholine-tuftsin conjugate of Formula 1. In some embodiments, the fungicidal composition comprises a fungicidal effective amount of the phosphorylcholine-tuftsin conjugate of Formula 2. In some embodiments, the fungicidal composition is devoid of a fungicide which is not a phosphorylcholine-tuftsin conjugate. In some embodiments, the fungicidal composition consists essentially of one or more phosphorylcholine-tuftsin conjugates of the invention as the fungicidal active ingredient. In some embodiments, the fungicidal composition consist essentially of one or more phosphorylcholine-tuftsin conjugates of the invention and a carrier.

In some embodiments, the fungicidal composition comprises a plurality (e.g. 2, 3, 4, 5, 10 including any range between) of the phosphorylcholine-tuftsin conjugates of the invention, wherein at least one phosphorylcholine-tuftsin conjugate is present at a fungicidal effective amount within the fungicidal composition.

In some embodiments, the fungicidal composition comprises a fungicidal effective amount of the phosphorylcholine-tuftsin conjugate of Formula 1, and further comprises the phosphorylcholine-tuftsin conjugate of Formula 2, and optionally comprises one or more functional derivative of the phosphorylcholine-tuftsin conjugate of Formula 2.

In some embodiments, the fungicidal composition comprises a fungicidal effective amount of the phosphorylcholine-tuftsin conjugate of Formula 2, and further comprises the phosphorylcholine-tuftsin conjugate of Formula 1, and optionally comprises one or more functional derivative of the phosphorylcholine-tuftsin conjugate of Formula 2.

In some embodiments, the carrier is a liquid carrier (e.g. a flowable carrier, such as an aqueous carrier, a semi-solid carrier, a semi-liquid carrier). In some embodiments, the carrier is a pharmaceutically acceptable carrier.

In some embodiments, the fungicidal composition is a liquid composition. In some embodiments, the fungicidal composition comprises the phosphorylcholine-tuftsin conjugate, and/or a salt thereof and/or a functional derivative thereof (e.g. a peptide having a similar structure and characterized by anti-fungal activity). In some embodiments, the fungicidal composition is a pharmaceutical composition comprising the phosphorylcholine-tuftsin conjugate and a pharmaceutically acceptable carrier. In some embodiments, the fungicidal composition is an anti-mold composition comprising the phosphorylcholine-tuftsin conjugate and an appropriate (e.g. food-grade) carrier.

In some embodiments, the salt is a pharmaceutically acceptable salt. In some embodiments, the salt is a cosmeceutically acceptable salt. In some embodiments, the salt is a food-grade salt.

In some embodiments, the fungicidal effective amount comprises a preservative effective concentration of one or more phosphorylcholine-tuftsin conjugates of the invention within the fungicidal composition. In some embodiments, the terms “preservative effective concentration” and “preservative effective amount” are used herein interchangeably and refer to a concertation of the phosphorylcholine-tuftsin conjugate(s) sufficient for the preservation activity of the fungicidal composition, as disclosed herein.

In some embodiments, the fungicidal effective amount comprises a concentration of one or more phosphorylcholine-tuftsin conjugates of the invention within the fungicidal composition of at least 1 nM, at least 10 nM, at least 100 nM, at least 500 nM, at least 1 μM, at least 2 μM, at least 5 μM, at least 10 μM, at least 15 μM, at least 20 μM, at least 30 μM, at least 40 μM, at least 50 μM, at least 75 μM, at least 100 μM, at least 150 μM, at least 200 μM, at least 400 μM, at least 500 μM, at least 600 μM, at least 1,000 μM, or any range or value therebetween. Each possibility represents a separate embodiment of the invention.

In some embodiments, the fungicidal effective amount comprises a concentration of at least one of the phosphorylcholine-tuftsin conjugates of the invention within the fungicidal composition of at least 1 nM, at least 10 nM, at least 100 nM, at least 500 nM, at least 1 μM, at least 2 μM, at least 5 μM, at least 10 μM, at least 15 μM, at least 20 μM, at least 30 μM, at least 40 μM, at least 50 μM, at least 75 μM, at least 100 μM, at least 150 μM, at least 200 μM, at least 400 μM, at least 500 μM, at least 600 μM, at least 1,000 μM, or any range or value therebetween. Each possibility represents a separate embodiment of the invention.

In some embodiments, the fungicidal effective amount comprises a concentration of one or more phosphorylcholine-tuftsin conjugates of the invention (e.g. conjugate of Formula 2) within the fungicidal composition of at least 1 ppm, at least 5 ppm, at least 10 ppm, at least 100 ppm, at least 500 ppm, at least 1000 ppm, or any range or value therebetween. Each possibility represents a separate embodiment of the invention.

In some embodiments, the fungicidal effective amount comprises a concentration of one or more phosphorylcholine-tuftsin conjugates of the invention (e.g. conjugate of Formula 1) within the fungicidal composition of at least 1 ppm, at least 5 ppm, at least 10 ppm, at least 100 ppm, at least 500 ppm, at least 1000 ppm, or any range or value therebetween. Each possibility represents a separate embodiment of the invention.

In some embodiments, a concentration of one or more phosphorylcholine-tuftsin conjugates of the invention within the fungicidal composition is between 5 ppm and 10% w/w, between 5 ppm and 99% w/w, between 5 ppm and 90% w/w, between 5 ppm and 95% w/w, between 5 ppm and 50% w/w, between 5 ppm and 60% w/w, between 5 ppm and 80% w/w, between 5 ppm and 5% w/w, between 5 ppm and 2% w/w, between 0.5 and 10% w/w, between 0.5 and 90% w/w, between 0.5 and 50% w/w, between 5 ppm and 0.5% w/w, including any range between.

Herein throughout, by “fungicidal composition”, it is further meant to refer to a formulation. As used herein, the term “formulation”, refer to a vehicle composition in the form of a solution, an emulsion, a lotion, a cream, a gel etc., that optionally further comprises physiologically acceptable carriers and/or excipients and optionally other chemical components such as cosmetically, cosmeceutically or pharmaceutically active agents (e.g., drugs). The formulation can optionally further comprise a carrier, and optionally additional active agents and/or additives.

In some embodiments, the fungicidal composition is a semi-solid or a gel at a temperature between 5 and 95° C. In some embodiments, the fungicidal composition is a flowable composition. In some embodiments, the fungicidal composition is a liquid or a flowable composition.

In some embodiments, a w/w concentration of the acceptable carrier within the fungicidal composition is between 50 and 99%, between 60 and 99%, between 70 and 99%, between 80 and 99%, between 90 and 99%, between 50 and 70%, between 70 and 90%, including any range therebetween.

In some embodiments, the fungicidal composition is a preservative composition. In some embodiments, the preservative composition of the invention is an aqueous solution comprising the active (or antimicrobial) ingredients substantially or fully dissolved therewithin. In some embodiments, the fungicidal composition of the invention is a ready to use composition or is in a form of a dilutable concentrate.

In some embodiments, the preservative composition of the invention, or the phosphorylcholine-tuftsin conjugate is characterized by sterilizing or preservation activity within a food matter (e.g. liquid, solid, or semi-liquid food matter).

In some embodiments, the concentration of one or more phosphorylcholine-tuftsin conjugate of the invention within the fungicidal composition is sufficient for substantially reducing or for preventing pathogen formation or pathogen growth on or within an edible matter (e.g. a food article) for at least 3 d, at least 4 d, at least 5 d, at least 6 d, at least 7 d, at least 8 d, at least 9 d, at least 10 d, at least 30 d, at least 60 d, including any range or value therebetween. Pathogen formation or pathogen growth can be determined visually, or by performing a microbiological test. In some embodiments, the concentration of one or more phosphorylcholine-tuftsin conjugate of the invention within the fungicidal composition is sufficient for substantially preventing spoilage of an edible matter (e.g. a food article) for at least 3 d, at least 4 d, at least 5 d, at least 6 d, at least 7 d, at least 8 d, at least 9 d, at least 10 d, at least 30 d, at least 60 d, including any range or value therebetween.

In some embodiments, reducing comprises at least a 10%, 20%, 30%, 40%, 50%, 60%, 70%, 80%, 90%, 95%, 99% or 100% reduction, including any range between. Each possibility represents a separate embodiment of the invention.

In some embodiments, increasing comprises at least a 10%, 20%, 30%, 40%, 50%, 60%, 70%, 80%, 90%, 95%, 99%, 100%, 150%, 200%, 300%, 400%, 500%, 1000%, or 10000% increase, including any range between. Each possibility represents a separate embodiment of the invention.

In some embodiments, the preservative composition of the invention is characterized by sterilizing or preservation activity within a liquid composition (such as an aqueous pharmaceutical or cosmeceutical composition) or on a surface upon contacting a fungicidal effective amount of the preservative composition with the liquid composition or with the surface under suitable conditions (such as a temperature between 1 and 40° C., for a time period between 1 minute and 14 days, including any range between). In some embodiments, the phosphorylcholine-tuftsin conjugate of the invention (e.g. the conjugate of Formula 1, or of Formula 2) is characterized by sterilizing or preservation activity within a liquid composition. In some embodiments, the sterilizing or preservation activity of the phosphorylcholine-tuftsin conjugate is at a fungicidal effective amount of the phosphorylcholine-tuftsin conjugate within the liquid composition, wherein fungicidal effective amount is as described hereinabove (e.g. at least 5 ppm). In some embodiments, the preservation activity refers to a preservative effectiveness, as determined by Preservative Effectiveness Testing (PET) according to USP<51>.

In some embodiments, the preservation activity refers to an antimicrobial activity. In some embodiments, the preservation activity refers to fungicidal activity.

In some embodiments, the liquid composition and/or the surface refers to liquid composition or surface prone of microbial infestation (such as articles comprising a water content of at least 10%, at least 50%, or between 10 and 99%, between 20 and 90%, between 20 and 80%, between 30 and 99%, including any range between).

In some embodiments, the fungicidal composition of the invention is formulated for use in a liquid composition or on a surface susceptible to microbial growth as a preservative. In some embodiments, the liquid composition is selected from a cosmeceutical article, a pharmaceutical article, an edible matter and/or a food article.

The term “antimicrobial” refers to a composition or compound capable of preventing the growth, inhibiting the growth, and/or controlling the growth of microorganisms; antimicrobial compounds include bactericides, bacteriostats, fungicides, fungistats, algaecides and algistats. The term “antimicrobial” encompasses preservatives and sterilization agents.

In some embodiments, the term “antimicrobial activity” is referred to as an ability to inhibit (prevent), reduce or retard bacterial growth, fungal growth, biofilm formation or eradicate living bacterial cells, or their spores, or fungal cells or viruses in a suspension or in a moist environment. In some embodiments, the term “antimicrobial activity” is referred to the ability of the of the phosphorylcholine-tuftsin conjugate(s) of the invention to inhibit (prevent), reduce or retard fungal growth, biofilm formation or eradicate living fungal cells, or their spores, in a suspension or in a moist environment. In some embodiments, the term “antimicrobial activity” is referred to the ability of the fungicidal effective amount of the phosphorylcholine-tuftsin conjugate(s) of the invention to inhibit (prevent), reduce or retard fungal growth, biofilm formation or eradicate living fungal cells, or their spores, in a liquid composition, in a living organism, or in a moist environment, wherein the fungicidal effective amount is as described hereinabove (e.g. at least 5 ppm) and refers to the concertation of the phosphorylcholine-tuftsin conjugate(s) of the invention (e.g. conjugates of Formula 1, or 2) within the liquid composition, in a target location within the living organism, or in a moist environment.

Herein, inhibiting or reducing or retarding the formation of load of a microorganism refers to inhibiting, reducing, or retarding growth of microorganisms and/or eradicating a portion or all of an existing population of microorganisms. Thus, formulations described herein can be used both in reducing the formation of microorganisms on or in an article, and in killing microorganisms in or on an article or a living tissue.

The microorganism can be, for example, a unicellular microorganism (prokaryotes, archaea, bacteria, eukaryotes, protists, fungi, algae, molds, yeast, euglena, protozoan, dinoflagellates, apicomplexa, trypanosomes, amoebae and the likes), or a multicellular microorganism.

In some embodiments, the microorganism comprises bacterial cells of bacteria such as, for example, Gram-positive and Gram-negative bacteria.

In some embodiments, the Gram-positive bacteria are Staphylococcus aureus, Staphylococcus epidermidis, and Bacillus cereus.

In some embodiments, the Gram-negative bacteria are Escherichia coli, Pseudomonas aeruginosa, and Burkholderia cepacia.

In some embodiments of the present invention, the microorganism is a mold, a yeast, or a fungus, including any combination thereof. In some embodiments of the present invention, the fungus is Candida albicans. In some embodiments of the present invention, the yeast is of genus Saccharomyces.

In some embodiments, the mold is Aspergillus brasiliensis.

In some embodiments, the term “fungi” including any grammatical form thereof refers to a pathogenic fungus or yeast. In some embodiments, the pathogenic fungus refers to a human pathogen.

In some embodiments, non-limiting examples of the fungus are selected from Penicillium, Magnaporthe, Ophiostoma, Cryphonectria, Fusarium, Ustilago, Alternaria, Cochliobolus, Aspergillus, Candida, Cryptococcus, Histoplasma, and Pneumocytis or any combination thereof.

In some embodiments, the pathogen is a fungus selected from, but not limited to: Alternaria spp. (e.g., Alternaria alternata, Alternaria solani); Aphanomyces spp. (e.g., Aphanomyces euteiches); Aspergillus spp. (e.g., Aspergillus niger, Aspergillus fumigatus); Atelia spp. (e.g., Atelia rolfsii); Aureobasidium spp. (e.g., Aureobasidium pullulans); Bipolaris spp. (e.g. Bipolaris zeicola, Bipolaris maydis); Botrytis spp. (e.g., Botrytis cinerea); Calonectria spp. (e.g., Calonectria kyotensis); Cephalosporium spp. (e.g., Cephalosporium maydis); Cercospora spp. (e.g., Cercospora medicaginis, Cercospora sojina, Colletotrichum coccodes, Colletotrichum fragariae, Colletotrichum graminicola); Coniella spp. (e.g., Coniella diplodiella); Colletotrichum spp.; Coprinopsis spp. (e.g., Coprinopsis psychromorbida); Corynespora spp. (e.g., Corynespora cassiicola; Curvularia spp. (e.g., Curvularia pallescens); Cylindrocladium spp. (e.g., Cylindrocladium crotalariae); Diplocarpon spp. (e.g., Diplocarpon earlianum); Diplodia spp. (e.g., Diplodia gossyina); Epicoccum spp. (e.g., Epicoccum nigrum); Erysiphe spp. (Erysiphe cichoracearum); Fusarium spp. (e.g., Fusarium graminearum, Fusarium oxysporum f. sp. fragariae, Fusarium oxysporum f. sp. tuberosi, Fusarium proliferatum var. proliferatum, Fusarium solani, Fusarium verticillioides, Fusarium clumorum, Fusarium oxysporum f. sp. radicis-lycopersici, Fusarium euwallaceae); Ganoderma spp. (e.g., Ganoderma boninense); Geotrichum spp. (e.g., Geotrichum candidum); Glomerella spp. (e.g., Glomerella tucumanensis); Guignardia spp. (e.g., Guignardia bidwellii); Kabatiella spp. (e.g., Kabatiella zeae); Leptosphaerulina spp. (e.g., Leptosphaerulina briosiana); Leptotrochila spp. (e.g., Leptotrochila medicaginis); Macrophomina spp. (e.g., Macrophomina phaseolina); Magnaporthe spp. (e.g., Magnaporthe grisea, Magnaporthe oryzae); Microsphaera spp. (e.g., Microsphaera manshurica); Monilinia spp. (e.g., Monilinia fructicola); Mucor spp.; Mycosphaerella spp. (e.g., Mycosphaerella juiensis, Mycosphaerella fragariae); Nigrospora spp. (e.g., Nigrospora oryzae); Ophiostoma spp. (e.g., Ophiostoma ulmi); Penicillium spp. (e.g., Penicillium digitatum); Peronospora spp. (e.g., Peronospora manshurica); Phakopsora (e.g., Phakopsora pachyrhizi); Phoma spp. (e.g., Phoma foveata, Phoma medicaginis, Phoma tracheiphila); Phomopsis spp (e.g. Phomopsis longicolla); Phytophthora spp. (e.g., Phytophthora cinnamomi, Phytophthora erythroseptica, Phytophthora fragariae, Phytophthora infestans, Phytophthora medicaginis, Phytophthora megasperma, Phytophthora palmivora); Podosphaera (e.g., Podosphaera leucotricha); Pseudopeziza spp. (e.g., Pseudopeziza medicaginis); Puccinia spp. (e.g., Puccinia graminis subsp. tritici (UG99), Puccinia striiformis, Puccinia recodita, Puccinia sorghi); Pyricularia spp. (Pyricularia grisea, Pyricularia oryzae); Pythium spp. (e.g., Pythium ultimum, Pythium aphanidermatum); Rhizoctonia spp. (e.g., Rhizoctonia solani, Rhizoctonia zeae); Rosellinia spp., Sclerotinia spp. (e.g., Sclerotinia minor; Sclerotinia sclerotiorum, Sclerotinina trifoliorum); Sclerotium spp. (e.g., Sclerotium rolfsii); Septoria spp. (e.g., Septoria glycines, Septoria lycoperski); Setomelanomma spp. (e.g., Setomelanomma turcica); Sphaerotheca spp. (e.g., Sphaerotheca macularis); Spongospora spp. (e.g., Spongospora subterranean); Stemphylium spp., Synchytrium spp. (e.g., Synchytrium endobioticum), Verticillium spp. (e.g., Verticillium albo-atrum, Verticillium dahliae). In some embodiments, the fungus comprises Penicillium digitatum and/or Geotrichum candidum.

In some embodiments, non-limiting examples of yeast are selected from Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae and Rhodotorula mucilaginosa or any combination thereof.

The term “biofilm”, as used herein, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism (prokaryotes, archaea, bacteria, eukaryotes, protists, fungi, algae, euglena, protozoan, dinoflagellates, apicomplexa, trypanosomes, amoebae and the likes), or cells of multicellular organisms in which case the biofilm can be regarded as a colony of cells (like in the case of the unicellular organisms) or as a lower form of a tissue.

In the context of the present embodiments, the cells are of microorganism origins, and the biofilm is a biofilm of microorganisms, such as bacteria and fungi. The cells of a microorganism growing in a biofilm may be physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion.

The term “antibiofilm” refers to the capacity of a substance to disturb the formation of a biofilm of bacterial, fungal and/or other cells, and/or to affect a reduction in the rate of buildup of a biofilm of bacterial, fungal and/or other cells, on a surface of a substrate.

As used herein, the term “preventing” in the context of antimicrobial or preservative, indicates that the growth rate of the microorganism cells is essentially nullified or is reduced by at least 20%, at least 30%, at least 40%, at least 50%, at least 60%, at least 70%, at least 80%, at least 90%, including any value therebetween, of the appearance of the microorganism in a comparable situation lacking the presence of the preservative of the invention or an article containing same. Each possibility represents a separate embodiment of the invention. Alternatively, preventing means a reduction to at least 15%, 10%, or 5% of the appearance of the microorganism cells in a comparable situation (e.g. an aqueous composition) lacking the presence of the preservative.

As used herein, the term “reducing” in the context of preservation activity or effectiveness, indicates that the growth rate (and or microbial loading expressed in CFU or CFU/ml) of the microorganism (including spores, cells, or biofilm thereof) is essentially reduced as compared to a similar article (or composition) devoid of the preservative of the invention. In some embodiments, the term “essentially reduced” comprises at least 2 times, at least 5 times, at least 10 times, at least 50 times, at least 100 times, at least 1000 times, at least 10.000 times, at least 100.000 times, at least 1000.000 times, between 10 and 1000.000 times, between 100 and 1000.000 times, between 1000 and 10.000.000 times, between 1000 and 1000.000 times, between 10.000 and 1000.000 times CFU/ml reduction, including any range between, as compared to the CFU/ml content of a similar composition devoid of the preservative of the invention (also referred to herein as the initial microbial loading). In some embodiments, the preservative substantially prevents microbial infestation of the article (or composition). In some embodiments, the article (or composition) comprising the preservative composition of the invention maintains its sterility upon exposing thereof to non-sterile conditions (e.g. ambient atmosphere comprising one or more microbes) for a time period between 1 day and 2 months, between 1 day and 1 month, between 1 and 20 d, between 1 and 60 d, between 1 and 50 d, between 10 and 60 d, between 1 and 10 d, between 10 and 50 d, between 10 and 40 d, between 10 and 30 d, including any range between.

In another aspect, there is provided a method for treating or preventing a disease or disorder associated with fungal loading in a subject, the method comprising administering to the subject an effective amount of the pharmaceutical composition of the invention.

By another aspect, there is provided a method reducing, and/or inhibiting fungal activity within a subject, the method comprising administering to the subject an effective amount of the pharmaceutical composition of the invention. In some embodiments, effective amount of the composition (or dosage) comprises an amount sufficient for reducing or ameliorating fungal load within a subject. In some embodiments, effective amount of the composition (or dosage) comprises an amount sufficient for prevention or treatment disease or a disorder associated fungal infection within a subject, wherein treatment encompasses alleviation of at least one symptom thereof, a reduction in the severity thereof, or inhibition of the progression thereof. In some embodiments, treating fungal infection comprises at least one of preventing, attenuating, and/or inhibiting a disease, a disorder, or a condition associated with fungal activity within a subject.

In another aspect, there is provided a method for reducing or preventing fungal load in an edible matter (e.g. a food product), the method comprising contacting the food product with a fungicidal effective amount of the fungicidal composition of the invention. In some embodiments, the effective amount of the fungicidal composition of the invention is so as to reducing pathogen load (e.g. CFU, CFU/cm³) on or within the food product at least by a factor of 10, at least by a factor of 30, at least by a factor of 50, at least by a factor of 60, at least by a factor of 65, at least by a factor of 70, at least by a factor of 100, at least by a factor of 200, at least by a factor of 400, at least by a factor of 800, at least by a factor of 1000, at least by a factor of 10,000, at least by a factor of 100,000, at least by a factor of 1,000,000, as compared to a similar food product devoid of a preservative (also referred herein as “a control”).

In another aspect of the invention, there is provided a food product comprising the phosphorylcholine-tuftsin conjugate of the invention. In some embodiments, the food product comprises an effective amount (e.g. fungicidal effective amount) of the phosphorylcholine-tuftsin conjugate of the invention. In some embodiments, the effective amount is sufficient for inhibition or reduction of fungal growth and/or fugal loading for at least 4 days, at least 5 days, at least 6 days, at least 7 days, including any range or value therebetween, as compared to a control. In some embodiments, the effective amount is sufficient for prolonging shelf-life of the food product, as compared to a control. In some embodiments, prolonging is by at least 4 days, at least 5 days, at least 6 days, at least 7 days, at least 10 days, at least 20 days, at least 30 days, including any range or value therebetween.

In some embodiments, the phosphorylcholine-tuftsin conjugate comprises at least one phosphorylcholine moiety or a derivative thereof and tuftsin or a derivative thereof covalently linked via a spacer. In some embodiments, the spacer comprises at least two amino acids. In some embodiments, the phosphorylcholine moiety or a derivative thereof is covalently linked to the spacer via a diazo group.

The term “phosphorylcholine (PC) conjugate” as used herein, refers to a phosphorylcholine moiety or a derivative thereof linked to tuftsin (T), optionally via a spacer.

As used herein, the term “tuftsin” refers to a tetra-peptide (threonine-lysine-proline-arginine, TKPR; SEQ ID NO: 1). Tuftsin may be synthesized chemically. Tuftsin is known for its phagocytosis-stimulating activity and augmentation of antigen presenting capacity of macrophages in-vitro and in-vivo. According to some embodiments, tuftsin may be considered as an immunomodulatory molecule.

The term “derivative of phosphorylcholine” as used herein, refers to any compound that is based off phosphorylcholine. The term “derivative of tuftsin” as used herein, refers to any polypeptide that is based off of TKPR. In some embodiments, the derivative retains the immunomodulatory effects of phosphorylcholine and/or tuftsin. In some embodiments, the derivative is a derivative comprising phosphorylcholine. In some embodiments, the derivative is a derivative comprising TKPR. A derivative is not merely a fragment of the polypeptide, nor does it have amino acids replaced or removed (an analog), rather it may have additional modification made to the polypeptide, such as a post-translational modification.

In some embodiments, the derivative of phosphorylcholine is selected from: 4-amino-phenyl-phosphocholine, 4-diazonio-phenyl-phosphorylcholine, 4-nitro-phenyl-phosphocholine and 12-(3-iodophenyl) dodecyl-phosphocholine among others. Each possibility is a separate embodiment of the invention.

The terms “tuftsin derivative”, “TD” and “tuftsin-derived carrier moiety” are interchangeable and refer to tuftsin (TKPR, SEQ ID NO: 1) attached to at least two additional amino acids which are independently selected. Non-natural amino acids, preferably non-charged and non-polar non-natural amino acids such as β-alanine-6-aminohexanoic acid and 5-aminopentanoic acid, may also be comprised in the tuftsin derivative. In some embodiments, the tuftsin derivative is Threonine-Lysine-Proline-Arginine-Glycine-Tyrosine (TKPRGY, SEQ ID NO: 2).

The term “moiety” as used herein refers to a part of a molecule, which lacks one or more atom(s) compared to the corresponding molecule. The term “moiety”, as used herein, further relates to a part of a molecule that may include either whole functional groups or parts of functional groups as substructures. The term “moiety” further means part of a molecule that exhibits a particular set of chemical and/or pharmacologic characteristics which are similar to the corresponding molecule.

The terms “linked” or “attached” as used herein refer to a bond between at least two molecules or moieties such that they are a single molecule. In some embodiments, the bond is a chemical bond. In some embodiments, the bond is a covalent bond. According to the principles of the present invention, the natural and non-natural amino-acids comprised in the tuftsin derivative are adjacent and attached to one another, while the at least one phosphorylcholine derivative is attached to the at least one tuftsin derivative either directly or indirectly via a spacer. In some embodiments, the at least one phosphorylcholine or derivative thereof is linked to the N-terminus of at least one tuftsin or derivative thereof. In some embodiments, the at least one phosphorylcholine or derivative thereof is linked to the C-terminus of at least one tuftsin or derivative thereof.

The term “spacer”, as used herein, refers to a connecting or otherwise bridging element between the tuftsin derivative and the PC derivative, typically linked by chemical methods or biological means thereto. Non-limiting examples of spacers include: amino acids, peptides, polypeptides, proteins, hydrocarbons and polymers among others. Each possibility is a separate embodiment of the invention. In some embodiments, the spacer is at least 2 amino acids. In some embodiments, the spacer is Glycine-Tyrosine. In some embodiments, the spacer is attached to the C-terminus of TKPR. In some embodiments, the spacer is attached to the N-terminus of TKPR.

In some embodiments, the phosphorylcholine-tuftsin conjugate described above comprises one phosphorylcholine derivative attached to one tuftsin derivative. In certain embodiments, the phosphorylcholine-tuftsin conjugate described above comprises a plurality of phosphorylcholine derivatives attached to a plurality of tuftsin derivatives. In certain embodiments, the phosphorylcholine-tuftsin conjugate described above comprises a plurality of tuftsin derivatives attached to one phosphorylcholine derivative. In certain embodiments, the phosphorylcholine-tuftsin conjugate described above comprises a plurality of phosphorylcholine derivatives attached to one tuftsin derivative.

In some embodiments, the phosphorylcholine-tuftsin conjugate described above comprises at least one phosphorylcholine or derivative thereof and the at least one tuftsin or derivative thereof separated by a spacer.

In some embodiments, the phosphorylcholine-tuftsin conjugate is represented by Formula 1:

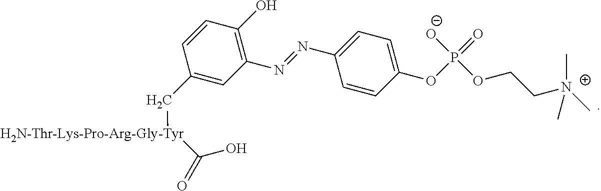

In some embodiments, the phosphorylcholine-tuftsin conjugate is in a form of a salt, comprising a counter ion. In some embodiments, the counter ion is a pharmaceutically acceptable ion. In some embodiments, the counter ion is any of chloride, acetate and trifluoroacetate or any combination thereof.

### Pharmaceutical Composition

In some embodiments, there is provided a pharmaceutical composition comprising one or more of the phosphorylcholine-tuftsin conjugates of the invention (e.g., conjugates of Formulae 1-5) and a pharmaceutically acceptable carrier. In some embodiments, the pharmaceutical composition comprises a therapeutically effective amount of one or more of the phosphorylcholine-tuftsin conjugates of the invention. In some embodiments, the pharmaceutical composition comprises a fungicidal effective amount of one or more of the phosphorylcholine-tuftsin conjugates of the invention.

In some embodiments, the pharmaceutical composition comprises a preservative effective amount, as disclosed hereinabove.

In some embodiments, the preservative effective amount is a concentration of one or more phosphorylcholine-tuftsin conjugates of the invention within the pharmaceutical composition of at least 1 nM, at least 10 nM, at least 100 nM, at least 500 nM, at least 1 μM, at least 2 μM, at least 5 μM, at least 10 μM, at least 15 μM, at least 20 μM, at least 30 μM, at least 40 μM, at least 50 M, at least 75 μM, at least 100 μM, at least 150 μM, at least 200 μM, at least 400 μM, at least 500 μM, at least 600 μM, at least 1,000 μM, or any range or value therebetween. Each possibility represents a separate embodiment of the invention.

In some embodiments, the preservative effective amount comprises a concentration of one or more phosphorylcholine-tuftsin conjugates of the invention (e.g. conjugate of Formula 2) within the pharmaceutical composition of at least 1 ppm, at least 5 ppm, at least 10 ppm, at least 100 ppm, at least 500 ppm, at least 1000 ppm, or any range or value therebetween. Each possibility represents a separate embodiment of the invention.

In some embodiments, the preservative effective amount comprises a concentration of one or more phosphorylcholine-tuftsin conjugates of the invention (e.g. conjugate of Formula 1) within the pharmaceutical composition of at least 1 ppm, at least 5 ppm, at least 10 ppm, at least 100 ppm, at least 500 ppm, at least 1000 ppm, or any range or value therebetween. Each possibility represents a separate embodiment of the invention.

In some embodiments, a concentration of one or more phosphorylcholine-tuftsin conjugates of the invention within the pharmaceutical composition is between 5 ppm and 10% w/w, between 5 ppm and 99% w/w, between 5 ppm and 90% w/w, between 5 ppm and 95% w/w, between 5 ppm and 50% w/w, between 5 ppm and 60% w/w, between 5 ppm and 80% w/w, between 5 ppm and 5% w/w, between 5 ppm and 2% w/w, between 0.5 and 10% w/w, between 0.5 and 90% w/w, between 0.5 and 50% w/w, between 5 ppm and 0.5% w/w, including any range between.

In some embodiments, a concentration of the phosphorylcholine-tuftsin conjugate of Formula 2, or of Formula 2A within the pharmaceutical composition or within the fungicidal composition of the invention is between 5 ppm and 10% w/w, 5 ppm and 50% w/w, between 5 ppm and 20% w/w, between 5 ppm and 30% w/w, between 5 ppm and 40% w/w, between 5 ppm and 2% w/w, between 5 ppm and about 1% w/w, between 10 ppm and 50% w/w, between 10 ppm and 20% w/w, between 10 ppm and 30% w/w, between 10 ppm and 10% w/w, between 10 ppm and 2% w/w, between 10 ppm and about 1% w/w, between 20 ppm and 50% w/w, between 20 ppm and 20% w/w, between 20 ppm and 30% w/w, between 20 ppm and 10% w/w, between 20 ppm and 2% w/w, between 20 ppm and about 1% w/w, between 5 ppm and 99% w/w, between 5 ppm and 90% w/w, between 5 ppm and 95% w/w, between 5 ppm and 50% w/w, between 5 ppm and 60% w/w, between 5 ppm and 80% w/w, between 5 ppm and 5% w/w, between 5 ppm and 2% w/w, between 0.5 and 10% w/w, between 0.5 and 90% w/w, between 0.5 and 50% w/w, between 5 ppm and 0.5% w/w, including any range between. In some embodiments, a concentration of the phosphorylcholine-tuftsin conjugate of Formula 2, or of Formula 2A within the pharmaceutical composition or within the fungicidal composition of the invention is at least the fungicidally effective concertation, as disclosed herein (e.g. at least about 10 ppm, or at least about 20 ppm, a concertation which experimentally induced a substantial inhibition of yeast activity in an aqueous medium, as disclosed in the Examples section).

In some embodiments, a concentration of the phosphorylcholine-tuftsin conjugate of Formula 1 within the pharmaceutical composition, or within the fungicidal composition of the invention is between 5 ppm and 10% w/w, 5 ppm and 50% w/w, between 5 ppm and 20% w/w, between 5 ppm and 30% w/w, between 5 ppm and 40% w/w, between 5 ppm and 2% w/w, between 5 ppm and about 1% w/w, between 10 ppm and 50% w/w, between 10 ppm and 20% w/w, between 10 ppm and 30% w/w, between 10 ppm and 10% w/w, between 10 ppm and 2% w/w, between 10 ppm and about 1% w/w, between 20 ppm and 50% w/w, between 20 ppm and 20% w/w, between 20 ppm and 30% w/w, between 20 ppm and 10% w/w, between 20 ppm and 2% w/w, between 20 ppm and about 1% w/w, between 5 ppm and 99% w/w, between 5 ppm and 90% w/w, between 5 ppm and 95% w/w, between 5 ppm and 50% w/w, between 5 ppm and 60% w/w, between 5 ppm and 80% w/w, between 5 ppm and 5% w/w, between 5 ppm and 2% w/w, between 0.5 and 10% w/w, between 0.5 and 90% w/w, between 0.5 and 50% w/w, between 5 ppm and 0.5% w/w, including any range between. In some embodiments, a concentration of the phosphorylcholine-tuftsin conjugate of Formula 1 within the pharmaceutical composition or within the fungicidal composition of the invention is at least the fungicidally effective concertation, as disclosed herein (e.g. at least about 10 ppm, or at least about 20 ppm, a concertation which experimentally induced a substantial inhibition of yeast activity in an aqueous medium, as disclosed in the Examples section).

In some embodiments, a combined concentration of the phosphorylcholine-tuftsin conjugate of Formula 1 and of the phosphorylcholine-tuftsin conjugate of Formula 2 (or of Formula 2A) within the pharmaceutical composition, or within the fungicidal composition of the invention is between 10 ppm and 50% w/w, between 10 ppm and 20% w/w, between 10 ppm and 30% w/w, between 10 ppm and 10% w/w, between 10 ppm and 2% w/w, between 10 ppm and about 1% w/w, between 20 ppm and 50% w/w, between 20 ppm and 20% w/w, between 20 ppm and 30% w/w, between 20 ppm and 10% w/w, between 20 ppm and 2% w/w, between 20 ppm and about 1% w/w, between 10 ppm and 99% w/w, between 10 ppm and 90% w/w, between 10 ppm and 95% w/w, between 10 ppm and 50% w/w, between 10 ppm and 60% w/w, between 10 ppm and 80% w/w, between 10 ppm and 5% w/w, between 10 ppm and 2% w/w, between 0.5 and 10% w/w, between 0.5 and 90% w/w, between 0.5 and 50% w/w, between 10 ppm and 0.5% w/w, including any range between.

In some embodiments, a concentration of the phosphorylcholine-tuftsin conjugate of Formula 1 within the pharmaceutical composition or within the fungicidal composition of the invention is between 0.5 and 10% w/w, between 0.5 and 50% w/w, between 0.5 and 5% w/w, between 0.5 and about 2% w/w, between 0.5 and about 1% w/w, between 10 ppm and 0.5% w/w, including any range between; and a concentration of the phosphorylcholine-tuftsin conjugate of Formula 2 or 2A within the pharmaceutical composition or within the fungicidal composition of the invention is between 10 ppm and 50% w/w, between 10 ppm and 20% w/w, between 10 ppm and 30% w/w, between 10 ppm and 10% w/w, between 10 ppm and 2% w/w, between 10 ppm and about 1% w/w, between 20 ppm and 50% w/w, between 20 ppm and 20% w/w, between 20 ppm and 30% w/w, between 20 ppm and 10% w/w, between 20 ppm and 2% w/w, between 20 ppm and about 1% w/w, between 10 ppm and 99% w/w, between 10 ppm and 90% w/w, between 10 ppm and 95% w/w, between 10 ppm and 50% w/w, between 10 ppm and 60% w/w, between 10 ppm and 80% w/w, between 10 ppm and 5% w/w, between 10 ppm and 2% w/w, between 0.5 and 10% w/w, between 0.5 and 90% w/w, between 0.5 and 50% w/w, between 10 ppm and 0.5% w/w, including any range between.

In some embodiments, a concentration of the phosphorylcholine-tuftsin conjugate of Formula 2 or 2A within the pharmaceutical composition or within the fungicidal composition of the invention is between 0.5 and 10% w/w, between 0.5 and 50% w/w, between 0.5 and 5% w/w, between 0.5 and about 2% w/w, between 0.5 and about 1% w/w, between 10 ppm and 0.5% w/w, including any range between; and a concentration of the phosphorylcholine-tuftsin conjugate of Formula 1 within the pharmaceutical composition or within the fungicidal composition of the invention is between 10 ppm and 50% w/w, between 10 ppm and 20% w/w, between 10 ppm and 30% w/w, between 10 ppm and 10% w/w, between 10 ppm and 2% w/w, between 10 ppm and about 1% w/w, between 20 ppm and 50% w/w, between 20 ppm and 20% w/w, between 20 ppm and 30% w/w, between 20 ppm and 10% w/w, between 20 ppm and 2% w/w, between 20 ppm and about 1% w/w, between 10 ppm and 99% w/w, between 10 ppm and 90% w/w, between 10 ppm and 95% w/w, between 10 ppm and 50% w/w, between 10 ppm and 60% w/w, between 10 ppm and 80% w/w, between 10 ppm and 5% w/w, between 10 ppm and 2% w/w, between 0.5 and 10% w/w, between 0.5 and 90% w/w, between 0.5 and 50% w/w, between 10 ppm and 0.5% w/w, including any range between.

In some embodiments, the pharmaceutical composition comprises the phosphorylcholine-tuftsin conjugate of Formula 2, including any salt or any functional derivative thereof. In some embodiments, the pharmaceutical composition comprises the phosphorylcholine-tuftsin conjugate of Formula 2, and further comprises at least a trace amount of any one of phosphorylcholine-tuftsin conjugates of Formulae 3-5, including any salt or any functional derivative thereof. In some embodiments, a w/w concertation of the phosphorylcholine-tuftsin conjugate of Formula 2 within the pharmaceutical composition is between about 1 ppm and about 10%, between about 5 ppm and about 10%, between about 5 ppm and about 0.5%, between about 5 ppm and about 1%, between about 5 ppm and about 2%, between about 10 ppm and about 10%, between about 10 ppm and about 200 ppm, between about 10 ppm and about 0.5%, between about 10 ppm and about 0.1%, between about 10 ppm and about 2%, between about 10 ppm and about 1%, between about 10 ppm and about 500 ppm, between about 10 ppm and about 2000 ppm, between about 10 ppm and about 1000 ppm, between about 10 ppm and about 100 ppm, between about 10 ppm and about 50 ppm, between about 5 ppm and about 50 ppm, between about 100 ppm and about 500 ppm, between about 100 ppm and about 1000 ppm, between about 1000 ppm and about 0.1%, between about 100 ppm and about 0.1%, between about 100 ppm and about 1%, between about 1000 ppm and about 1%, between about 0.1 and about 10% w/w, including any range between.

In some embodiments, the pharmaceutical composition comprises a pharmaceutically active agent and a carrier, wherein the pharmaceutically active agent consist essentially of one or more of the phosphorylcholine-tuftsin conjugates of the invention. phosphorylcholine-tuftsin conjugate as the only pharmaceutically active ingredient. In some embodiments, the pharmaceutical composition is substantially devoid of any additional pharmaceutically active ingredient. In some embodiments, the pharmaceutically active agent consist essentially of the phosphorylcholine-tuftsin conjugate of Formula 2, and optionally one or more functional derivative thereof. In some embodiments, the pharmaceutically active agent consist essentially of the phosphorylcholine-tuftsin conjugate of Formula 2 optionally including one or more functional derivative thereof; and of the phosphorylcholine-tuftsin conjugate of Formula 1. In some embodiments, the pharmaceutically active agent consist essentially of the phosphorylcholine-tuftsin conjugate of Formula 2 and of the phosphorylcholine-tuftsin conjugate of Formula 1, wherein a w/w ratio between the phosphorylcholine-tuftsin conjugate of Formula 1 and the phosphorylcholine-tuftsin conjugate of Formula 2 is between 10:1 and 10.000:1, between 10:1 and 1000:1, between 100:1 and 10.000:1, between 200:1 and 10.000:1, between 200:1 and 1000:1, including any range between.

In some embodiments, the pharmaceutical composition comprises the phosphorylcholine-tuftsin conjugate of Formula 2 including any salt or any functional derivative thereof, and further comprises the phosphorylcholine-tuftsin conjugate of Formula 1. In some embodiments, a w/w concentration the phosphorylcholine-tuftsin conjugate of Formula 1 within the pharmaceutical composition is between 0.01 and 10%, between 0.05 and 10%, between 0.1 and 10%, between 0.1 and 1%, between 0.1 and 0.5%, between 0.5 and 1%, between 0.5 and 1.5%, between 0.5 and 2%, between about 0.8 and about 1%, between 0.5 and 10%, between 0.5 and 5%, between 1 and 10%, including any range between.

In some embodiments, a w/w concentration the phosphorylcholine-tuftsin conjugate of Formula 1 within the pharmaceutical composition is between 0.1 and 10%, between 0.5 and 2%, between 0.1 and about 1%, between 0.1 and about 2%, including any range between; and wherein a w/w concentration the phosphorylcholine-tuftsin conjugate of Formula 2 within the pharmaceutical composition is between about 5 ppm and about 10%, between about 5 ppm and about 0.5%, between about 5 ppm and about 1%, between about 5 ppm and about 2%, between about 10 ppm and about 10%, between about 10 ppm and about 200 ppm, between about 10 ppm and about 0.5%, between about 10 ppm and about 0.1%, between about 10 ppm and about 2%, between about 10 ppm and about 1%, between about 10 ppm and about 500 ppm, between about 10 ppm and about 2000 ppm, between about 10 ppm and about 1000 ppm, between about 10 ppm and about 100 ppm, between about 10 ppm and about 50 ppm, between about 5 ppm and about 50 ppm, between about 100 ppm and about 500 ppm, between about 100 ppm and about 1000 ppm, between about 1000 ppm and about 0.1%, between about 100 ppm and about 0.1%, between about 100 ppm and about 1%, between about 1000 ppm and about 1%, between about 0.1 and about 10% w/w, including any range between.

In some embodiments, a w/w concentration the phosphorylcholine-tuftsin conjugate of Formula 1 within the pharmaceutical composition between 0.1 and about 2%, including any range between; and wherein a w/w concentration the phosphorylcholine-tuftsin conjugate of Formula 2 within the pharmaceutical composition is between about 5 ppm and about 2%.

In some embodiments, a w/w concentration the phosphorylcholine-tuftsin conjugate of Formula 1 within the pharmaceutical composition between 0.1 and about 2%, including any range between; and wherein a w/w concentration the phosphorylcholine-tuftsin conjugate of Formula 2 within the pharmaceutical composition is between about 5 ppm and about 200 ppm.

In some embodiments, the pharmaceutical composition comprises a preservative effective amount of at least one phosphorylcholine-tuftsin conjugate of any one of Formulae 1-5. In some embodiments, the pharmaceutical composition comprises a preservative effective amount of the phosphorylcholine-tuftsin conjugate of Formula 1, or a preservative effective amount of the phosphorylcholine-tuftsin conjugate of Formula 2.

In some embodiments, the pharmaceutical composition is an ophthalmic composition. In some embodiments, the pharmaceutical composition is a liquid, a flowable composition, a semi-solid, as semi-liquid, a foam, or a gel.

In one aspect of the invention, there is a pharmaceutical composition comprising (i) one or more phosphorylcholine-tuftsin conjugates as disclosed hereinabove, (ii) between 0.1 and 5% weight per weight (w/w) of a viscosity enhancer, and (iii) between 0.1 and 5% w/w of a mucoadhesive polymer. In some embodiments, the pharmaceutical composition is a liquid composition. In some embodiments, the pharmaceutical composition is formulated for ocular administration. In some embodiments, the terms “ophthalmic composition” and “pharmaceutical composition” are used herein interchangeably.

In some embodiments, the ophthalmic or the pharmaceutical composition is a liquid at a temperature between-5 and 95° C. In some embodiments, the ophthalmic composition is a liquid under physiological conditions (e.g. a temperature between 2° and 40° C., a pH between 2 and 8, etc.) In some embodiments, the ophthalmic composition is an aqueous composition. In some embodiments, the ophthalmic composition is a solution (e.g. an aqueous solution). In some embodiments, the components of the ophthalmic composition are substantially soluble within the solution. In some embodiments, the ophthalmic composition is substantially devoid of any particulate matter. In some embodiments, the ophthalmic composition is substantially devoid of particles having a particle size greater than 200 nm, 300 nm, 400 nm, 500 nm, 600 nm, 700 nm, 800 nm, 900 nm, 1 μm, including any range or value therebetween. In some embodiments, the ophthalmic composition is a clear solution. In some embodiments, the ophthalmic composition is an opaque solution.

In some embodiments, the ophthalmic composition is characterized by transmittance of more than 80%, more than 85%, more than 90%, more than 95%, more than 97%, more than 99%, wherein the transmittance is measured at wavelengths ranging from 200 nm to 1000 nm. One skilled in the art will appreciate that transmittance is an indication for optical clarity of the ophthalmic composition (e.g. in a form of a solution).

In some embodiments, the ophthalmic composition is a colored solution. In some embodiments, the colored solution is characterized by a slight yellow to red color.

In some embodiments, the ophthalmic composition comprises a solvent. In some embodiments, the solvent is an aqueous solvent. In some embodiments, the ophthalmic composition comprises between 50 and 99%, between 50 and 60%, between 60 and 70%, between 70 and 80%, between 80 and 85%, between 85 and 90%, between 90 and 95%, between 90 and 92%, between 92 and 95%, between 95 and 97%, between 97 and 99%, w/w of the solvent including any range or value therebetween.

In some embodiments, a w/w concentration of a viscosity enhancer within the pharmaceutical composition is between 0.1 and 5%, between 0.1 and 0.2%, between 0.2 and 0.3%, between 0.3 and 0.5%, between 0.4 and 0.6%, between 0.5 and 0.7%, between 0.7 and 0.8%, between 0.8 and 1%, between 1 and 2%, between 2 and 3%, between 3 and 5%, including any range or value therebetween. In some embodiments, a w/w concentration of the viscosity enhancer within the pharmaceutical composition is between 0.4 and 0.6%.

As used herein, a “viscosity enhancer” refers to any substance that increases the viscosity of the solution to be administered to the eye. In some embodiments, the viscosity enhancer increases viscosity of an aqueous solution.

Non-limiting examples of viscosity enhancers include but are not limited to hydroxypropyl methyl cellulose (HPMC), hydroxy methyl cellulose, hydroxy ethyl cellulose, cellulose, polyalcohol polyvinylpyrrolidone, polyethyleneglycol, polypropyleneglycol, polyacrylate, poly(2-hydroxyethyl methacrylate), and polyvinylalcohol or any combination thereof.

Additional viscosity enhancers are well known in the art, such as poloxamer, galactomannan, hydrolyzed galactomannan, glucomannan, hydrolyzed glucomannan, guar gum, xanthan gum, nanocrystalline cellulose, cyclodextrins, poly(cyclodextrins), dextran, dextrin, starch, heparin, beta-glucan, mannan, and levan or any combination thereof.

In some embodiments, the pharmaceutical composition comprises a mucoadhesive polymer. In some embodiments, a w/w concentration of the mucoadhesive polymer within the pharmaceutical composition is between 0.1 and 5%, between 0.1 and 0.2%, between 0.2 and 0.3%, between 0.3 and 0.5%, between 0.4 and 0.6%, between 0.5 and 0.7%, between 0.7 and 0.8%, between 0.8 and 1%, between 1 and 2%, between 2 and 3%, between 3 and 5%, including any range or value therebetween. In some embodiments, a w/w concentration of the mucoadhesive polymer within the pharmaceutical composition is between 1 and 1.5%.

In some embodiments, the mucoadhesive polymer is an ionic mucoadhesive polymer. In some embodiments, the mucoadhesive polymer is a cationic polymer, an anionic polymer or both.

Non-limiting examples of mucoadhesive polymers include, but are not limited to alginic acid, chitosan, hyaluronic acid, carboxymethylcellulose, pectin, fucoidan, dermatan sulfate, chondroitin sulfate, polycarbophil-cysteine, polyoxyethylene glycol ester, heparan sulfate, keratin sulfate, and gelatin, including any salt, any copolymer, or any combination thereof.

In some embodiments, the mucoadhesive polymer is alginic acid (or alginate) and/or a salt thereof.

In some embodiments, a w/w ratio between the mucoadhesive polymer and the viscosity enhancer within the ophthalmic composition is between 1:1 and 10:1, between 1:1 and 2:1, between 2:1 and 4:1, between 2:1 and 3:1, between 3:1 and 5:1, between 3:1 and 4:1, between 5:1 and 7:1, between 7:1 and 10:1, including any range or value therebetween.

In some embodiments, a w/w ratio between the mucoadhesive polymer and the phosphorylcholine-tuftsin conjugate within the ophthalmic composition is between 5:1 and 1:5, between 5:1 and 3:1, between 3:1 and 2:1, between 2:1 and 1:1, between 1:1 and 1:2, between 1:2 and 1:4, between 1:4 and 1:5, including any range or value therebetween.

In some embodiments, the ophthalmic composition comprises up to between 0.1 and 10% w/w of a tonicity regulator.

In some embodiments, the ophthalmic composition comprises an effective amount of a preservative. In some embodiments, the effective amount is a preservative effective amount. In some embodiments, the effective amount is an antimicrobial effective amount. In some embodiments, the effective amount of the preservative within the ophthalmic composition of the invention is reduced relative to the label claim of the preservative (i.e. the effective concentration of the specific preservative as prescribed by the relevant regulatory authority).

In some embodiments, the effective amount of the preservative within the ophthalmic composition of the invention is reduced by at least 10%, at least 50%, at least 70%, at least 2 times, at least 5 times, at least 10 times, at least 50 times, at least 100 times, at least 500 times, or between about 2 and about 20 times, between about 2 and about 15 times, between about 2 and about 10 times, between about 5 and about 20 times, between about 5 and about 15 times, including any range or value therebetween, wherein reduced is relative to the label claim of the preservative. In some embodiments, the ophthalmic composition comprising a reduced amount of the preservative, wherein reduced is relative to effective amount of the preservative within a similar composition devoid of phosphorylcholinetuftsin conjugate.

In some embodiments, the effective amount of the preservative is determined based on a PET test, according to USP <51>, or any alternative PET test.

In some embodiments, the ophthalmic composition comprises between 0.001 and 1% w/w of a preservative, including any range between. In some embodiments, the ophthalmic composition comprises at least 0.001% w/w and below the label claim of the preservative, wherein below the label claim encompasses a concentration of the preservative being reduced by at least 10%, at least 50%, at least 70%, at least 2 times, at least 5 times, at least 10 times, at least 50 times, at least 100 times, at least 500 times, or between about 2 and about 20 times, between about 2 and about 15 times, between about 2 and about 10 times, between about 5 and about 20 times, between about 5 and about 15 times, including any range or value therebetween, relative to the label claim of the preservative.

In some embodiments, a w/w concentration of the preservative within the ophthalmic composition is between 0.001 and 1%, between 0.001 and 0.1%, between 0.001 and 0.01%, between 0.001 and 0.02%, between 0.001 and 0.018%, between 0.001 and 0.015%, between 0.002 and 0.018%, between 0.002 and 0.015%, between 0.003 and 0.018%, between 0.003 and 0.015%, between 0.05 and 0.1%, between 0.1 and 0.2%, between 0.2 and 0.3%, between 0.3 and 0.4%, between 0.4 and 0.5%, between 0.5 and 0.7%, between 0.7 and 1%, including any range or value therebetween. In some embodiments, the preservative is suitable for use in an ophthalmic composition. In some embodiments, the preservative is or comprises a quaternary ammonium cation, such as benzalkonium (BAK) including any salt thereof, for example benzalkonium chloride. In some embodiments, the quaternary ammonium cation is a pharmaceutically acceptable compound. In some embodiments, the quaternary ammonium cation comprises a pharmaceutically acceptable counter anion.

The inventors surprisingly found a synergistic preservative activity of the phosphorylcholine-tuftsin conjugate (specifically, phosphorylcholine-tuftsin conjugate of Formula 1) and BAK, resulting in significantly reduced concentration of BAK in the composition of the invention required for the sufficient preservative activity, relative to the label claim of BAK in ophthalmic preparations (0.02% w/w).

In some embodiments, the quaternary ammonium cation is represented by Formula NR₄⁺, wherein each R is independently selected from an alkyl group, an aryl group, an alkyl-aryl group, and wherein each of alkyl group, aryl group, and alkyl-aryl group is optionally substituted by one or more substituents each independently selected from —NO₂, —CN, —OH, oxo, imino, —CONH₂, —CONR′₂, —CNNR′₂, —CSNR′₂, —CONH—OH, —CONH—NH₂, —NHCOR′, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂—NH(C₁-C₆ alkyl), —N(C₁-C₆ alkyl)₂, C₁-C₆ alkoxy, C₁-C₆ haloalkoxy, hydroxy (C₁-C₆ alkyl), hydroxy (C₁-C₆ alkoxy), alkoxy (C₁-C₆ alkyl), alkoxy (C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(—O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy (C₁-C₆ alkyl), hydroxy (C₁-C₆ alkoxy), alkoxy (C₁-C₆ alkyl), alkoxy (C₁-C₆ alkoxy), C₁-C₆ alkyl-NR′₂, C₁-C₆ alkyl-SR′, or a combination thereof.

As used herein, the term “alkyl” describes an aliphatic hydrocarbon including straight chain and branched chain groups. The alkyl group has between 1 to 50 carbon atoms, 1-30 carbon atoms, or 1-20 carbon atoms. Whenever a numerical range; e.g., “1-30”, is stated herein, it implies that the group, in this case the alkyl group, may contain 1, 2 carbon atoms, 3 carbon atoms, 4 carbon atoms, 5 carbon atoms, 6 carbon atoms, 7 carbon atoms, 10 carbon atoms, 15 carbon atoms, 20 carbon atoms, etc., up to and including 30 carbon atoms. In some embodiments, the alkyl group is a short alkyl comprising between 1 and 10, or between 1 and 6 carbon atoms, including any range between. In some embodiments, the alkyl group is a long alkyl having at least 10 carbon atoms, or between 10 and 30 carbon atoms including any range between. The alkyl can be substituted or unsubstituted, as defined herein.

The term “alkyl”, as used herein, also encompasses saturated or unsaturated hydrocarbon, hence this term further encompasses alkenyl and alkynyl.

In some embodiments, the quaternary ammonium is a polymer comprising one or more quaternary ammonium cations of Formula [symbol] , wherein each R is independently as disclose hereinabove, and wherein a wavy bond represents the attachment point to the polymeric backbone. Exemplary polymeric quaternary ammonium cations include but are not limited to Polyquaternium (e.g. Polyquaternium-2, Polyquaternium-80, Polyquaternium-11, Polyquaternium-52, Polyquaternium-17, etc.).

In some embodiments, the quaternary ammonium cation and/or salt thereof is selected from, without being limited thereto, benzyldimethyldodecylammonium chloride, didecyldimethylammonium chloride, dodecyltrimethylammonium chloride, hexadecyltrimethylammonium chloride, Sodium Cocamidopropyl PG-Dimonium Chloride Phosphate (Cola Lipid C), Laurdimoniumhydroxypropyl decylglucoside chloride (SugaQuat L-1010), Berberine, Berberrubine, Berberis vulgaris root extract, Isopropylbenzyl butylnorberberine iodide, Palmatine, Jatrorrhizine, Coptisine, Ungeremine, Epiberberine, Pseudoberberine, Stepharanine, Sinapine, and any combination thereof.

Additional preservatives are well-known in the art. Non-limiting examples of preservatives include but are not limited to, chlorobutanol, sodium perborate, and stabilized oxychloro complex (SOC) or any combination thereof.

In some embodiments, the preservative as disclosed herein is the only preservative in the ophthalmic composition of the invention. In some embodiments, the preservative within the ophthalmic composition is a benzalkonium, including any salt and any derivative thereof.

In some embodiments, the term “derivative” refers to a stereoisomer (e.g. enantiomer, and/or diastereomer) and/or a structural isomer (e.g. an alkylated, an animated, a hydroxylated, a carboxylated derivative), an ester, a tautomer, and/or any prodrug or precursor of the above disclosed compounds. In some embodiments, the term “derivative” refers to a structural derivative having a similar (or an enhanced) antimicrobial activity.

In some embodiments, the ophthalmic composition of the invention comprises below 0.02%, below 0.019%, or below 0.018% w/w of BAK or any derivative of BAK. In some embodiments, the ophthalmic composition of the invention comprises between 0.001 and 0.018%, between 0.001 and 0.015%, between 0.001 and 0.01%, between 0.001 and 0.02%, between 0.001 and 0.018%, between 0.001 and 0.015%, between 0.002 and 0.018%, between 0.002 and 0.015%, between 0.003 and 0.018%, between 0.003 and 0.015% w/w, including any range or value therebetween.

In another aspect, a w/w ratio (also referred to herein as “synergistically effective ratio”) between the phosphorylcholine-tuftsin conjugate and the quaternary ammonium cation within the pharmaceutical composition is between about 1000:1 and about 1:1 between about 500:1 and 1:1, between about 700:1 and 1:1, between about 100:1 and 1:1, between about 50:1 and 1:1, between about 1000:1 and 10:1, between about 100:1 and 10:1, between about 500:1 and 10:1, between about 100:1 and 20:1, between about 50:1 and 10:1, between about 50:1 and 20:1, between about 10:1 and 5:1, between about 10:1 and 1:1, including any range between.

In some embodiments, the quaternary ammonium cation is BAK. In some embodiments, the antimicrobial effective amount of BAK within the pharmaceutical composition (i.e. aqueous composition) is below 0.2 mg/ml, below 0.18 mg/ml, below 0.15 mg/ml, between 0.01 and 0.19 mg/ml, between 0.01 and 0.18 mg/ml, between 0.01 and 0.15 mg/ml, between 0.01 and 0.1 mg/ml, between 0.02 and 0.18 mg/ml, between 0.02 and 0.15 mg/ml, between 0.015 and 0.18 mg/ml, between 0.015 and 0.15 mg/ml, between 0.03 and 0.18 mg/ml, between 0.03 and 0.15 mg/ml, including any range between.

In some embodiments, the ophthalmic composition comprises or consists essentially of: an aqueous solution (e.g. a buffered aqueous solution), between 0.1 and 5 w/w, between 0.1 and 2 w/w, between 0.1 and 3 w/w, between 5 ppm and 10% w/w of one or more phosphorylcholine-tuftsin conjugate of the invention, between 0.5 and 1.5% w/w of the mucoadhesive polymer (e.g. alginate), between 0.3 and 0.8% w/w of the viscosity enhancer (e.g. HPMC), about 4% w/w of the tonicity regulator (e.g. mannitol), and the antimicrobial effective amount of the preservative (e.g. BAK). Exemplary composition is as described hereinbelow. In some embodiments, the ophthalmic composition comprises or consists essentially of: an aqueous solution (e.g. a buffered aqueous solution), between 0.1 and 2 w/w of one or more phosphorylcholine-tuftsin conjugate of the invention (i.e. the conjugate of Formula 1, and further comprising between 5 ppm and 0.1% w/w of the conjugate of Formula 2), between 0.5 and 1.5% w/w of the mucoadhesive polymer (e.g. alginic acid), between 0.3 and 0.8% w/w of the viscosity enhancer (e.g. HPMC), about 4% w/w of the tonicity regulator (e.g. mannitol), and between 0.001 and 0.019% w/w of the preservative (e.g. BAK). Exemplary composition is described hereinbelow.

In some embodiments, the ophthalmic composition comprises or consists essentially of: an aqueous solution (e.g. a buffered aqueous solution), between 0.5 and about 1% w/w of the conjugate of Formula 1, and further comprising between 5 ppm and 200 ppm of the conjugate of Formula 2, between 0.5 and 1.5% w/w of the mucoadhesive polymer (e.g. alginic acid), between 0.3 and 0.8% w/w of the viscosity enhancer (e.g. HPMC), about 4% w/w of the tonicity regulator (e.g. mannitol), and between 0.001 and 0.019% w/w of the preservative (e.g. BAK).

In another aspect of the invention, there is provided an ophthalmic composition comprising or consisting essentially of: an aqueous solution (e.g. a buffered aqueous solution), between 0.5 and 1.5% w/w of the mucoadhesive polymer (e.g. alginic acid), between 0.3 and 0.8% w/w of the viscosity enhancer (e.g. HPMC), about 4% w/w of the tonicity regulator (e.g. mannitol), and an effective amount of the preservative of the invention, (i.e. a combination between a quaternary ammonium cation, such as BAK and the phosphorylcholine-tuftsin conjugate of Formula 1). In some embodiments, the effective amount of the preservative (also used herein as “the preservative effective concentration”) is reduced, compared to a preservative effective concentration of the quaternary ammonium cation within a similar composition devoid of PC phosphorylcholine-tuftsin conjugate wherein “reduced” is as described hereinabove.

In some embodiments, the effective amount of the preservative within the ophthalmic composition of the invention corresponds to a concentration of the quaternary ammonium cation within between 0.01 and 0.19 mg/ml, between 0.01 and 0.18 mg/ml, between 0.01 and 0.15 mg/ml, between 0.01 and 0.1 mg/ml, between 0.02 and 0.18 mg/ml, between 0.02 and 0.15 mg/ml, between 0.015 and 0.18 mg/ml, between 0.015 and 0.15 mg/ml, between 0.03 and 0.18 mg/ml, between 0.03 and 0.15 mg/ml, including any range between. In some embodiments, the effective amount of the preservative within the ophthalmic composition of the invention further comprises phosphorylcholine-tuftsin conjugate, wherein the w/w ratio of phosphorylcholine-tuftsin conjugate to the quaternary ammonium cation is as described herein. In some embodiments, the term “corresponds to”, when related to the concentration of the quaternary ammonium means that the concentration of the phosphorylcholine-tuftsin conjugate may vary, however the w/w ratio between the phosphorylcholine-tuftsin conjugate and the quaternary ammonium cation within the ophthalmic composition of the invention is as described hereinabove.

In some embodiments, the ophthalmic composition consists essentially of pharmaceutical grade constituents. In some embodiments, the entire constituents of the ophthalmic composition are pharmaceutical grade compounds. In some embodiments, the ophthalmic composition is sterile. In some embodiments, the effective amount of the preservative within the ophthalmic composition of the invention is sufficient for maintain the sterility of the ophthalmic composition upon exposure thereof to non-sterile condition (e.g. ambient atmosphere comprising one or more microbes), wherein maintain is for a time period ranging between 1 and between 1 day and 2 months, between 1 day and 1 month, between 1 and 20 d, between 1 and 60 d, between 1 and 50 d, between 10 and 60 d, between 1 and 10 d, between 10 and 50 d, between 10 and 40 d, between 10 and 30 d, including any range between. The term “sterility” as used herein, refers inter alia to the microbial load of a composition (e.g. ophthalmic composition of the invention) or article according to the sterility requirements as recorded in US Pharmacopoeia, or in European Pharmacopoeia, respectively.

In some embodiments, the pH of the ophthalmic composition disclosed herein is between 6 and 8, between 6 and 6.5, between 6.5 and 7, between 7 and 7.5, between 7.5 and 8, including any range or value therebetween.

In some embodiments, the ophthalmic composition is characterized by a viscosity between 30 and 150 cps, between 50 and 120 cps, between 1 and 5 cps, between 5 and 10 cps, between 10 and 15 cps, between 15 and 20 cps, between 20 and 30 cps, between 30 and 40 cps, between 40 and 50 cps, between 50 and 60 cps, between 60 and 70 cps, between 70 and 80 cps, between 80 and 90 cps, between 90 and 100 cps, between 100 and 110 cps, between 110 and 120 cps, including any range or value therebetween.

In some embodiments, the ophthalmic composition is characterized by a tonicity between 200 and 600 mOsmols/kg, between 200 and 500 mOsmols/kg, between 200 and 300 mOsmols/kg, between 300 and 350 mOsmols/kg, between 350 and 400 mOsmols/kg, between 400 and 450 mOsmols/kg, between 450 and 500 mOsmols/kg, between 500 and 550 mOsmols/kg, between 550 and 600 mOsmols/kg, including any range or value therebetween. In some embodiments, the ophthalmic composition is characterized by a tonicity between 250 and 450 mOsmols/kg.

In some embodiments, the pharmaceutical composition comprises the phosphorylcholine conjugate and at least one other ingredient, as well as any product which results, directly or indirectly, from combination, complexation, or aggregation of any two or more of the ingredients, from dissociation of one or more of the ingredients, or from other types of reactions or interactions of one or more of the ingredients. Accordingly, the term “pharmaceutical composition” as used herein may encompass, inter alia, any composition made by admixing a pharmaceutically active amount of the phosphorylcholine-tuftsin conjugate and one or more pharmaceutically acceptable carriers. In some embodiments, the pharmaceutical composition comprises a pharmaceutically acceptable carrier, diluent or excipient.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein, including any range between.

In some embodiments, the pharmaceutical composition is an aqueous solution. In some embodiments, the aqueous solution is a buffered solution. In some embodiments, the aqueous solution comprises a pharmaceutically acceptable salt. In some embodiments, the buffered solution comprises a phosphate buffer. In some embodiments, the aqueous solution comprises water for injection, which is well-known in the art.

Among suitable buffer components or buffering agents that may be employed in the aqueous solution, are those conventionally used in pharmaceutical compositions. In some embodiments, the pharmaceutical composition is formulated for topical and/or systemic administration.

An embodiment of the invention relates to phosphorylcholine-tuftsin conjugate of the present invention, presented in unit dosage form and prepared by any of the methods well known in the art of pharmacy. In one embodiment, the unit dosage form is in the form of a tablet, capsule, lozenge, wafer, patch, ampoule, vial or pre-filled syringe. In one embodiment, pharmaceutical compositions are formulated in a compatible pharmaceutical carrier are prepared, placed in an appropriate container, and labeled for treatment of an indicated condition.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In one embodiment, the composition of the present invention is administered in the form of a pharmaceutical composition comprising at least one of the active components of this invention together with a pharmaceutically acceptable carrier or diluent. In another embodiment, the composition of the invention can be administered either individually or together in any conventional oral, parenteral or transdermal dosage form.

For topical application, the phosphorylcholine-tuftsin conjugate of the invention, or combination thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

The composition also includes incorporation of the active agent into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such composition will influence the physical state, solubility, stability, rate of in vivo release, and rate of in vivo clearance.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In some embodiments, pharmaceutically acceptable salt includes but is not limited to alkali metal, alkaline earth metal and/or ammonium salts, as well as citrate, phosphate, borate, lactate and the like salts and mixtures thereof. Conventional organic buffers, such as Goode's buffer and the like, may also be employed.

In some embodiments, the pharmaceutical composition comprises a therapeutically effective amount of one or more of the phosphorylcholine-tuftsin conjugate of the invention. The term “therapeutically effective amount” refers to the amount of the conjugate effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition. In some embodiments, the term “therapeutically effective amount” and the term “pharmaceutically effective amount” are used herein interchangeably.

### Method

In another aspect, there is a method for treating or preventing a disease or a disorder associated with fungal infection of a subject, the method comprising administering to the subject a therapeutically effective amount of the fungicidal composition of the invention. In some embodiments, the method is for inhibiting, reducing or decreasing fungal load in a subject in need thereof. In some embodiments, the composition is for use in inhibiting, reducing or decreasing fungal activity in a subject in need thereof. In some embodiments, the fungal activity is related to an infection by one or more fungal species.

In some embodiments, the subject is afflicted with a fungal infection. In some embodiments, the disclosed invention is directed to a fungicidal composition for use in inhibiting, reducing or decreasing fungal activity. In some embodiments, fungal activity comprises any activity selected from the group consisting of: proliferation, fungicide resistance, cell communication or quorum sensing, biofilm production, toxin production or secretion, and combination thereof.

In some embodiments, the method further comprises a pre-step performed before the step of administration. In some embodiments, pre-step comprises determining of a subject afflicted with a fungal infection. In some embodiments, determining comprises determining of fungal activity. In some embodiments, determining is performed within a sample derived from the subject. Fungal activity can be assayed using any common method, non-limiting examples of which include, but are not limited to, spectrophotometry, drug resistance assays using selective substrates, bioluminescence assay, liquid chromatography and mass-spectrometry or others, which would be apparent to one of ordinary skill in art.

As used herein, the terms “treatment” or “treating” of fungal infection and/or a disease or a disorder associated therewith, encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, treating fungal infection comprises at least one of preventing, attenuating, and/or inhibiting a disease, a disorder, or a condition associated with fungal activity within a subject.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, and pig. In one embodiment, the subject is a human. Human subjects may also include fetuses.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

As used herein, the term “anti-fungal activity” refers to the ability to inhibit, prevent, reduce or retard any one of: growth, protein production rate, protein production yield, protein synthesis, messenger RNA translation rate, biofilm formation of at least one fungus, and/or to eradicate living fungal cells, or their spores. In some embodiments, anti-fungal activity is achieved on a surface or in a moist environment. In some embodiments, inhibiting or reducing or retarding the formation of load of a fungus refers to inhibiting, reducing or retarding growth of a fungus and/or eradicating a portion or all of an existing population of fungi.

Methods for determining reduced protein synthesis, protein production rates, and yields, are common and would be apparent to one of ordinary skill in the art, non-limiting example of which is shown hereinbelow, using a Luciferase reporter assay.

In some embodiments, inhibit, prevent, retard, or reduce is by at least 5%, at least 15%, at least 25%, at least 40%, at least 50%, at least 65%, at least 75%, at least 85%, at least 90%, at least 95%, at least 99% compared to control, or any value or range therebetween. In some embodiments, modifying is by 1-5%, 7-15%, 10-25%, 20-40%, 35-50%, 45-65%, 55-75%, 70-85%, 80-90%, 87-95%, or 92-100% compared to control. Each possibility represents a separate embodiment of the invention.

In some embodiments, reducing comprises at least a 10%, 20%, 30%, 40%, 50%, 60%, 70%, 80%, 90%, 95%, 99% or 100% reduction. Each possibility represents a separate embodiment of the invention.

As used herein, the terms “administering”, “administration”, and like terms refer to any method which, in sound medical practice, delivers a composition containing an active agent to a subject in such a manner as to provide a therapeutic effect. In some embodiments, the administering is ocular or intraocular.

A person skilled in the art will appreciate that eye drops in particular, and to an extent ointments as well, will not perfectly reach the site of inflammation. As such the dose will need to be increased or decreased as determined by a skilled artisan to compensate for the mode of administration. Doses by intraocular injection will more directly reach the site of inflammation and again the dose administered will need to be adjusted accordingly.

In some embodiments, the amount (dose) of a composition to be administered will, of course, be dependent on the subject being treated, in the medical condition being treated for, the severity of the affliction, the manner of administration, the judgment of the prescribing physician, etc.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the fungicidal composition of the present invention is administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the conjugates, or any derivative or combination thereof, is the amount of the mentioned herein conjugates necessary for the in vivo measurable expected biological effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In some embodiments, the daily dose (i.e. the amount of the phosphorylcholine-tuftsin conjugate per day) is between 1 and 2000 μg, between 100 and 1000 ng, between 200 and 2000 μg, between 1 and 10 μg, between 10 and 50 μg, between 50 and 100 μg, between 100 and 200 μg, between 200 and 300 μg, between 300 and 400 μg, between 400 and 500 μg, between 500 and 600 μg, between 600 and 700 μg, between 700 and 800 μg, between 800 and 900 μg, between 900 and 1000 μg, between 1000 and 1100 μg, between 1100 and 1300 μg, between 1300 and 1500 μg, between 1500 and 1800 μg, between 1800 and 2000 μg, including any range or value therebetween.

In some embodiments, the fungicidal composition is administered 1, 2, 3, 4, 5, 6, 8, 9, 10 times a day, including any range therebetween.

The pharmaceutical composition may further comprise additional pharmaceutically active or inactive agents such as, but not limited to, an anti-fungal agent, an antioxidant, a buffering agent, a bulking agent, a surfactant, an anti-inflammatory agent, an anti-viral agent, a chemotherapeutic agent and anti-histamine.

According to an embodiment of the present invention, the pharmaceutical composition described herein above is packaged in a packaging material and identified in print, in or on the packaging material, for use in the treatment of a disease or disorder, as described herein.

According to another embodiment of the present invention, the pharmaceutical composition is packaged in a packaging material and identified in print, in or on the packaging material, for use in monitoring a disease or disorder, as described herein.

Products of the present invention may, if desired, be presented in a pack or dispenser device, such as an U.S. Food and Drug Administration (FDA) approved kit, which may contain one or more unit dosage forms containing the disclosed composition. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the FDA for prescription drugs or of an approved product insert.

In some embodiments, the kit contains a single dosage form, wherein the dosage form contains a daily dose of the disclosed composition. In some embodiments, the kit contains a plurality of dosage forms. In some embodiments, the kit contains a plurality of dosage forms, wherein the plurality of dosage forms are equivalent to a daily dose of the disclosed composition.

It is understood that the composition of the present invention may be administered in conjunction with other drugs, including other anti-fungal drugs.

### General

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed formulation, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Materials used in the manufacture of the exemplary ophthalmic compositions are as follows: TRS (Drug Substance, Investigational grade); Monobasic sodium phosphate, dihydrate (Buffering Agent, USP/NF); Dibasic sodium phosphate dihydrate (Buffering Agent, USP/NF); Benzalkonium Chloride (Preservative, USP/NF); Sodium Alginate (mucoadhesive, Ph Eur); Hypromellose (Viscosity Enhancer, USP/NF); Sodium Hydroxide and/or Hydrochloric Acid (pH Adjusting agent, USP/NF); Water for Injection (Solvent, USP/NF); Mannitol (Tonicity regulator, USP).

Drug Substance [TRS]: is a trifluoroacetate [TFA] salt of phosphorylcholine-tuftsin conjugate represented by Formula 1 (as shown herein). Molecular formula: C43H68N13O13P, Appearance: Yellow to orange solid. Solubility: >40 mg/ml in water.

Packaging of the ophthalmic compositions: single use media bottles (PETG) with HDPE screw caps. After filling the closure is sealed with tamper-proof labels. Bottles and closures are purchased radiation-sterilized and non-pyrogenic. Storage conditions: −20° C.±5° C. Long term stability study of [TRS] at −20° C.±5° C. and an accelerated stability study at +5° C.±3° C., started recently for 36 months. Available stability data at −20° C.±5° C. proved that the drug substance is stable for 6 months if stored under these conditions.

Exemplary pharmaceutical formulation of the invention is as follows: TRS (about 1% w/w), Phosphate buffer (10 mM), HPMC (about 0.5% w/w), BAK (about 0.02% w/w), Mannitol (about 1.7% w/w).

### Example 1

The phosphorylcholinetuftsin conjugate of Formula 2A, and the phosphorylcholinetuftsin conjugates represented by Formulae 3A, 4 and 5 have been synthesized by reacting TRS with acetaldehyde, as disclosed herein below.

In brief, 50 mg of TRS was dissolved in 2.5 mL of water and 50 equivalents of acetaldehyde were added (at room temperature). Then, the pH was adjusted to 6.8. The solution was shaken over the weekend and analyzed by an analytical HPLC. Various phosphorylcholine tuftsin conjugates have been detected in the reaction mixture, such as phosphorylcholine tuftsin conjugates of Formula 2A and of Formula 3A, and further phosphorylcholine tuftsin conjugates represented by any of Formulae 4 and/or 5. The reaction mixture was purified by a preparative HPLC, to isolate the phosphorylcholine tuftsin conjugate of Formula 3A. However, it was impossible to isolate the phosphorylcholine tuftsin conjugate of Formula 2A in form of a pure compound using a preparative HPLC separation method.

Subsequently, the inventors successfully obtained the phosphorylcholine tuftsin conjugate of Formula 2A (chemical purity above 90%), via partial hydrolysis of the phosphorylcholine tuftsin conjugate of Formula 3A. The phosphorylcholine tuftsin conjugate of Formula 3A was dissolved in 0.1% formic acid in water and the pH was adjusted to 2.7. After 30 min, the solution was lyophilized. The crude reaction mixture was purified by preparative HPLC to obtain the phosphorylcholine tuftsin conjugate of Formula 2A.

The chemical structures of the synthesized phosphorylcholine tuftsin conjugates have been confirmed by LC-MS and optionally by 1D-, 2D-NMR.

The phosphorylcholine tuftsin conjugate of Formula 2A has been found to exhibit a solid biological activity, and a sufficient chemical stability. In contrast, phosphorylcholine tuftsin conjugate of Formula 3A undergoes hydrolysis (is converted into PC of Formula 2A at acidic pH). PC of any one of Formulae 4 or 5 (an additional derivative of the PC of Formula 2) was characterized by a reduced chemical stability, as compared to PC of Formula 2.

### Example 2

The inventors have surprisingly discovered a synergistic preservative activity of the combination of the phosphorylcholine tuftsin conjugate of Formula 1 (TRS) and benzalkonium chloride. In brief, 1.0% w/w TRS formulations based on the constituents of the TRS formulation disclosed below, with three various concentration of benzalkonium chloride have been prepared. Subsequently, the solutions have been subjected to the Preservative Effectiveness Testing (PET) according to USP<51>.

TRS formulation that contained only 0.003% w/w benzalkonium chloride (corresponding to 15% of the label claim) passed PET test. Furthermore, TRS formulation with even lower concentration of benzalkonium chloride of 0.001% (corresponding to 5% label claim) showed a solid synergistic antimicrobial effect. A similar formulation without benzalkonium chloride didn't show any significant antimicrobial effect, pointing out the synergistic effect of both benzalkonium chloride and TRS in the pharmaceutical composition of the invention.

The inventors will perform a similar experiment with additional phosphorylcholine tuftsin conjugates disclosed herein. Based on the fungicidal effect demonstrated for both phosphorylcholine tuftsin conjugates of Formulae 2 and 1 (see Example 3), it is presumed that additional phosphorylcholine tuftsin conjugates (e.g. conjugates of Formulae 2 and/or 3) will exhibit a similar synergistic antimicrobial effect.

### Example 3

In a preliminary study, inhibition of yeast cellular respiration in an aqueous suspension was observed as reduced fermentation activity (resulted in decreased gas formation) in the presence of TRS (phosphorylcholinetuftsin conjugate (PC) of Formula 1), whereas a control suspension without TRS exhibited normal yeast cellular respiration associated with substantial gas formation. Yeast fermentation activity was correlated to the formation of bubbles within the aqueous suspension.

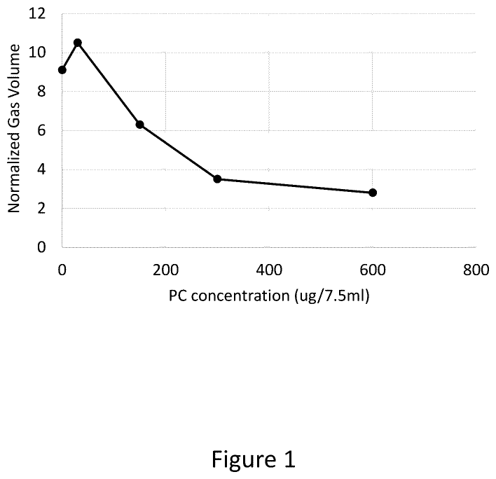

In brief, varying amount of 1% w/w solution of TRS was added to an aqueous suspension containing yeasts (S. cerevisiae) and a sufficient amount of sucrose, to obtain aqueous compositions containing 4 ppm, 20 ppm, 40 ppm, and 80 ppm of TRS. The gas formation (fermentation activity) of the aqueous suspension with TRS (hereinafter, “the sample”) was quantified by evaluating the volume of the released gas and compared to a similar aqueous yeast suspension devoid of TRS (hereinafter, “the control”). The results of this Experiment are presented in FIG. 1.

Surprisingly, it has been observed that the samples with 40 ppm and 80 ppm of TRS are almost completely devoid of fermentation activity (no bubbles were observed), while the control was characterized by substantial fermentation activity, as deduced from vigorous gas formation therewithin. As shown in FIG. 1, a sample with 20 ppm TRS (20 μg/ml) already exhibited a significant reduction of fermentation activity, compared to control. Accordingly, it has been postulated that TRS is effective in inhibiting or reducing fungal load and/or fungal activity.

Further, the phosphorylcholine tuftsin conjugate (PC) of Formula 2A synthesized as disclosed herein, has been tested to evaluate its antifungal activity. The inventors performed a similar experiment, as disclosed above, using varying concentrations of the phosphorylcholine tuftsin conjugate of Formula 2 (i.e. 6.7 ppm, 20 ppm, 40 ppm, and 80 ppm).

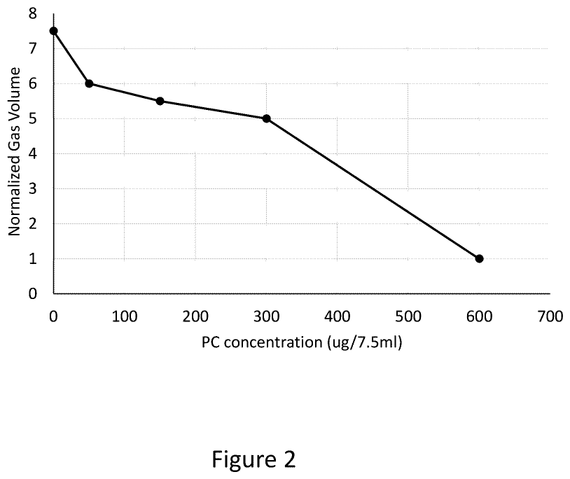

The results of the above experiment (yeast activity plotted versus concentration of each of the PC in the suspension) are represented in FIG. 2. FIG. 2 demonstrates a significant reduction of the yeast activity even at the lowest concentration of about 7 ppm of the conjugate of Formula 2. The yeast activity is further reduced with increasing concentrations of the conjugate, and is almost completely eliminated at 80 ppm of the conjugate of Formula 2.

Based on this experiments, the inventors presume that the phosphoryl tuftsin conjugates of the invention will show a solid fungicidal activity against additional fungal/yeast, and/or mold species.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A phosphorylcholine-tuftsin conjugate, including any functional derivative thereof, a salt thereof, or both; wherein said phosphorylcholine-tuftsin conjugate is 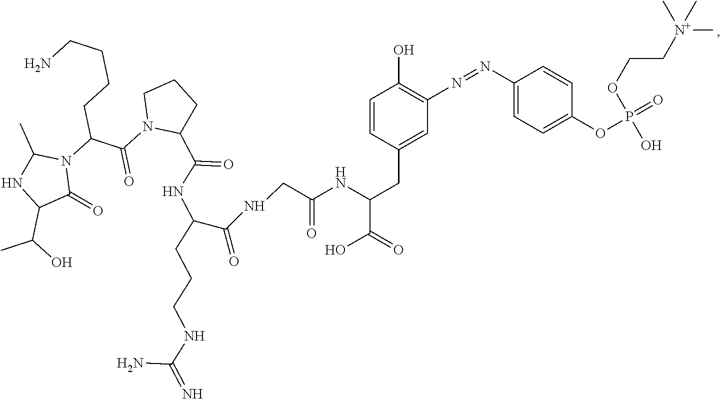 [SMILES: CC(O)C1NC(C)N(C(CCCCN)C(=O)N2CCCC2C(=O)NC(CCCNC(=N)N)C(=O)NCC(=O)NC(Cc2ccc(O)c(N=Nc3ccc(OP(=O)(O)OCC[N+](C)(C)C)cc3)c2)C(=O)O)C1=O]
wherein said functional derivative thereof is not a phosphorylcholine-tuftsin conjugate of Formula 1.

2. The phosphorylcholine-tuftsin conjugate of claim 1, wherein said phosphorylcholine-tuftsin conjugate is characterized by an antifungal activity.

3. The phosphorylcholine-tuftsin conjugate of claim 1, wherein said salt is a pharmaceutically acceptable salt; and wherein said phosphorylcholine-tuftsin conjugate is characterized by a chemical purity of at least 95%.

4. The phosphorylcholine-tuftsin conjugate of claim 1, wherein said functional derivative is:

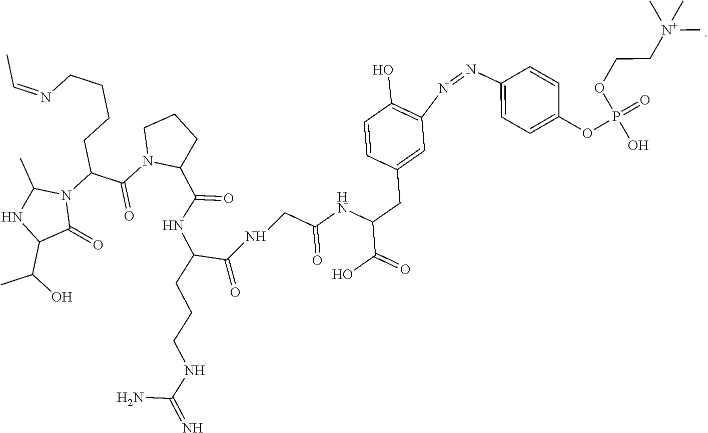 [SMILES: C/C=N\CCCCC(C(=O)N1CCCC1C(=O)NC(CCCNC(=N)N)C(=O)NCC(=O)NC(Cc1ccc(O)c(/N=N\c2ccc(OP(=O)(O)OCC[N+](C)(C)C)cc2)c1)C(=O)O)N1C(=O)C(C(C)O)NC1C]

5. A pharmaceutical composition comprising the phosphorylcholine-tuftsin conjugate of claim 1, and a pharmaceutically acceptable carrier.

6. The pharmaceutical composition of claim 5, comprising a fungicidal effective amount of the phosphorylcholine-tuftsin conjugate; optionally wherein the fungicidal effective amount is at least about 5 μg/ml.

7. (canceled)

8. The pharmaceutical composition of claim 5, wherein a concentration of the phosphorylcholine-tuftsin conjugate within said composition is between about 5 ppm and about 2% w/w.

9. The pharmaceutical composition of claim 5, wherein the pharmaceutical composition further comprises an additional phosphorylcholine-tuftsin conjugate of Formula 1:

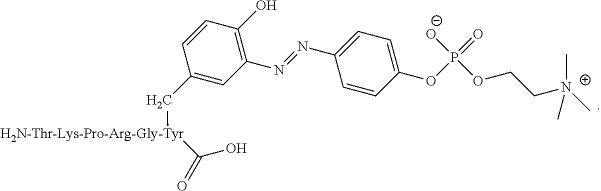

10. The pharmaceutical composition of claim 9, wherein a concentration of the additional phosphorylcholine-tuftsin conjugate within said pharmaceutical composition is between 5 ppm and 30% w/w.

11. The pharmaceutical composition of claim 9, wherein a concentration of the additional phosphorylcholine-tuftsin conjugate within said pharmaceutical composition is between 0.002 and 10% w/w.

12. The pharmaceutical composition of claim 9, wherein a concentration of the additional phosphorylcholine-tuftsin conjugate within said pharmaceutical composition is between about 0.5 and about 1% w/w.

13. The pharmaceutical composition of claim 5, wherein said pharmaceutically acceptable carrier is an aqueous composition comprising between 0.1 and 5% weight per weight (w/w) of a viscosity enhancer, and between 0.1 and 5% w/w of a mucoadhesive polymer.

14. The pharmaceutical composition of claim 13, wherein a w/w ratio between said mucoadhesive polymer and said viscosity enhancer is between 1:1 and 10:1; wherein said mucoadhesive polymer is an ionic mucoadhesive polymer; optionally wherein said ionic mucoadhesive polymer is selected from alginic acid, chitosan, polyacrylate, hyaluronic acid, carboxymethylcellulose, pectin, and gelatin including any salt, any copolymer, or any combination thereof; wherein said viscosity enhancer comprises hydroxy methyl cellulose, hydroxy ethyl cellulose, cellulose, hydroxypropyl methyl cellulose, polyvinylpyrrolidone, polyethyleneglycol, polypropyleneglycol, poly(2-hydroxyethyl methacrylate), and polyvinylalcohol, including any salt, any copolymer, or any combination thereof.

15. (canceled)

16. (canceled)

17. The pharmaceutical composition of claim 14, wherein said aqueous composition further comprises up to 10% w/w of a tonicity regulator, wherein said viscosity enhancer comprises hydroxy methyl cellulose, and wherein said mucoadhesive polymer comprises alginic acid, including any salt thereof; and wherein said pharmaceutical composition is an ophthalmic composition formulated for ocular administration.

18. (canceled)

19. A fungicidal composition comprising a fungicidal effective amount of one or more phosphorylcholine-tuftsin conjugate, a salt thereof, a functional derivative thereof, or any combination thereof; and further comprising a pharmaceutically acceptable carrier.

20. The fungicidal composition of claim 19, wherein said one or more phosphorylcholine-tuftsin conjugate comprises at least one phosphorylcholine moiety or a derivative thereof and tuftsin or a derivative thereof covalently linked via a spacer; and wherein said one or more phosphorylcholine-tuftsin conjugate comprises (i) a phosphorylcholine-tuftsin conjugate of Formula 1; (ii) the phosphorylcholine-tuftsin conjugate of any one of claims 1 to 4; or both (i) and (ii).

21. (canceled)

22. The fungicidal composition of claim 19, wherein a w/w of the pharmaceutically acceptable carrier within the fungicidal composition is between 10 and 99.99%; and wherein said pharmaceutically acceptable carrier is an aqueous formulation.

23.-27. (canceled)
